SEQUENCE LISTING (27 sequences)

SEQ ID NO: 1 - DNA, 2426 bp, Murinae gen. sp.
  CDS (650)..(1639)
aaagagaggctgacctggaaatcagttccacagcactggaccagaggagaaccaggtagaagaaacccctgaacttgcccaaggaggtaagctgacaggttgggaggctggggaggtggcagggggtgggcacctgaagctgaggagagcgagggttagcagggttggtttgccttctggaagtttcctttttctcccgagacacacacctacctgatctttcccagcttctatggctgccttcccctctctcccaccaggctttagcccccagccacagtcagcagcaagaaagggaaccgatcacagctacttcctattacctgtgaggctacagaggaaaggggccctagaaagcagaggggaaattcccgtgggaagtcaacttggtctcccccagagacacaggatggcaaacgtaataactgcagcttaatatcccgcccaaggctaagtcaataattgctctgggcagttctggcaggtggctwgagcaagcggtggtgaagagcagacagctttggactgaggacaggagagaaccagagaggatgtgagcagtcagaactctccggaaagagggtctgcaggctgtgtgcactgcaggtggggagctcttgaccctacacctttccccccatcttccagaatgaccccagactggcacagttccttgatcctcacggcctacatcctcatctttcttactgggctccctgccaacctgctggccctgcgggccttcatgggccgggttcgccagcctcagcctgcccctgtgcacatcctcctgcttaatctgaccctggcggacttgctcctgttgctgctgctgcccttccggatcgtggaagcagcatccaacttccgctggtacctaccaaagatcgtgtgcgcgctgacaggcttcggcttctacagcagcatctactgcagcacgtggctgctggcgggcatcagcatggaacgctacctgggagtggccttcccggtgcagtacaagttatcccgccggccactgtatggagtgatcgctgctctggtggcctggatcatgtcctttggccactgcaccatcgtcatcatcgttcagtacctgaactcaactgagcaggtgggcactgagaaccaaataacctgctacgagaacttcacccaagagcagctggatgtggtactgcccgtacgactggagctgtgcctggtcctgtttttcgttcccatggcagtcaccatcttctgttattggcgcttcgtgtggatcatgctcacgcagccccacgttggggctcagaggcgacgccgggcagtgggcctggctgttgtgacgcttcttaatttcctggtgtgctttggaccctacaacatgtcccacctggtggggttctacctgaggcagagcccctcgtggcgggtggaggctgtggtgttcagttccctcaatgccagcctggatccattattgttctacttctcctcctccgtggtgcgcagagcttttgggaaaggtttgctactgatccgcaatcctgcctcctctatgctgggcaggggagccaaagagacagtggaggggaccaagatggacaggggtggaagtcaagcagaaggggtacagagttctgaatttgtcaccgagtagagagtcccccggatcttcccgagcatttgggtcacccagaagttggtctggttagcagagctcctgagacttggtaaaagataaaagattaagaaagaaagaaagaaagaaagaaagaaagaaagaaagaaagaaagaaagaaagaaagaaagaaagacacctccacccatctgccactgccctctcagctcagcagaaccacttgactgaattttcctgcccagagaagcatagctccaggatacaagaaggaatggctacacccttacactgtattttcacagtggggtcaggggctcagaagcaaggtgactaataggggtgcagtaataaaagtaatttttttcctcgagcttggaaatgacacctagagacagtgatctggctggtgatgctggaaagaagccaatggaaaagatggggggagatggggggaagaagaggaatgccgggaaagctctaagccacagccttgaggcaggggctgagggcgggcaacacacagggctcccatctctggtgatcttggcctttctatatccgattgagctgtcctttgtacatgtgctccgctgattttcatattttggtcttgtctccactgttatgccgggatctgggtcacatgcttatcctctgtgtcaccagtcactgggtctaggagatgtcttggtacccgtctgtctgacattgacaatgacctgaaaggaaggattaaaaagcaaaatctggtttggcaggtaaaaataaagattatgaaagtctgaagcaaaaaaaaaaaaa

SEQ ID NO: 2 - protein, 330 aa, Murinae gen. sp.
MTPDWHSSLILTAYILIFLTGLPANLLALRAFMGRVRQPQPAPVHILLLNLTLADLLLLLLLPFRIVEAASNFRWYLPKIVCALTGFGFYSSIYCSTWLLAGISMERYLGVAFPVQYKLSRRPLYGVIAALVAWIMSFGHCTIVIIVQYLNSTEQVGTENQITCYENFTQEQLDVVLPVRLELCLVLFFVPMAVTIFCYWRFVWIMLTQPHVGAQRRRRAVGLAVVTLLNFLVCFGPYNMSHLVGFYLRQSPSWRVEAVVFSSLNASLDPLLFYFSSSVVRRAFGKGLLLIRNPASSMLGRGAKETVEGTKMDRGGSQAEGVQSSEFVTE

SEQ ID NO: 3 - DNA, 1616 bp, Homo sapiens
  CDS (88)..(1077)
ctaaagctctgttcctctctcctggaagcttgcagacctcccttcagaaccaatcccaagaagccacctatccggaacaacacaaggatgctgccggactggaagagctccttgatcctcatggcttacatcatcatcttcctcactggcctccctgccaacctcctggccctgcgggcctttgtggggcggatccgccagccccagcctgcacctgtgcacatcctcctgctgagcctgacgctggccgacctcctcctgctgctgctgctgcccttcaagatcatcgaggctgcgtcgaacttccgctggtacctgcccaaggtcgtctgcgccctcacgagttttggcttctacagcagcatctactgcagcacgtggctcctggcgggcatcagcatcgagcgctacctgggagtggctttccccgtgcagtacaagctctcccgccggcctctgtatggagtgattgcagctctggtggcctgggttatgtcctttggtcactgcaccatcgtgatcatcgttcaatacttgaacacgactgagcaggtcagaagtggcaatgaaattacctgctacgagaacttcaccgataaccagttggacgtggtgctgcccgtgcggctggagctgtgcctggtgctcttcttcatccccatggcagtcaccatcttctgctactggcgttttgtgtggatcatgctctcccagccccatgtgggggcccagaggcggcgccgagccgtggggctggctgtggtgacgctgctcaatttcctggtgtgcttcggaccttacaacgtgtcccacctggtggggtatcaccagagaaaaagcccctggtggcggtcaatagccgtggtgttcagttcactcaacgccagtctggaccccctgctcttctatttctcttcttcagtggtgcgcagggcatttgggagagggctgcaggtgctgcggaatcagggctcctccctgttgggacgcagaggcaaagacacagcagaggggacaaatgaggacaggggtgtgggtcaaggagaagggatgccaagttcggacttcactacagagtagcagtttccctggaccttcagaggtcgcctgggttacacaggagctgggaagcctgggagaggcggagcaggaaggctcccatccagattcagaaatccttagacccagcccaggactgcgactttgaaaaaaatgcctttcaccagcttggtatcccttcctgactgaattgtcctactcaaaggagcataagtcagagatgcacgaagaagtagttaggtatagaagcacctgccgggtgtggtggctcatgcctataatcccagaacattgggaggctgaggcaggtggatcacttgaggtcgggagattgagaacatcctggtcaacatgggaaaaccccgtctctactaaaaatacaaaaaaattagctgggcatggtggcacatgcctataatcccagctactctggaggctgaggcaggagaatccttgaacccgggagttggaggttgcagtgagctgagatcacgccactgcactccagcctagcgacagagcaagactccatttaaaaaaaaaaaaaaaaaaaaaaa

SEQ ID NO: 4 - protein, 330 aa, Homo sapiens [sequence omitted]

SEQ ID NO: 5 - protein, 8 aa, Murinae gen. sp.
MTPDWHSS

SEQ ID NO: 6 - protein, 18 aa, Murinae gen. sp.
RIVEAASNFRWYLPKIVC

SEQ ID NO: 7 - protein, 17 aa, Murinae gen. sp.
QYLNSTEQVGTENQITC

SEQ ID NO: 8 - protein, 20 aa, Murinae gen. sp.
QVGTENQITCYENFTQEQLD

SEQ ID NO: 9 - protein, 19 aa, Murinae gen. sp.
CYENFTQEQLDVVLPVRLE

SEQ ID NO: 10 - protein, 15 aa, Murinae gen. sp.
SHLVGFYLRQSPSWR

SEQ ID NO: 11 - protein, 18 aa, Homo sapiens
KIIEAASNFRWYLPKVVC

SEQ ID NO: 12 - protein, 17 aa, Homo sapiens
QYLNTTEQVRSGNEITC

SEQ ID NO: 13 - DNA, 21 bp, Murinae gen. sp.
cagactggcacagttccttga

SEQ ID NO: 14 - DNA, 18 bp, Murinae gen. sp.
cagatgggtggaggtgtc

SEQ ID NO: 15 - DNA, 21 bp, Homo sapiens
ggcacagttccttgatcctca

SEQ ID NO: 16 - DNA, 21 bp, Homo sapiens
gctcttgggtgaagttctcgt

SEQ ID NO: 17 - DNA, 27 bp, Homo sapiens
gagccacgtgctgcagtagatgctgct

SEQ ID NO: 18 - DNA, 28 bp, Homo sapiens
ctgccaacctcctggccctgcgggcctt

SEQ ID NO: 19 - DNA, 29 bp, Homo sapiens
gtaccagcggaagttcgacgcagcctcga

SEQ ID NO: 20 - DNA, 24 bp, Homo sapiens
gtcctttggtcactgcaccatcgt

SEQ ID NO: 21 - protein, 330 aa, thrombin receptor
LTSPWLTLFMPSVYTIVFIVSLPLNVLAIAVFVLRMKVKKPAVVYMLHLAMADVLFVSVLPFKISYYFSGTDWQFGSGMCRFATAAFYGNMYASIMLMTVISIDRFLAVVYPIQSLSWRTLGRANFTCVVIWVMAIMGVVPLLLKEQTTRVPGLNITTCHDVLSENLMQGFYSYYFSAFSAIFFLVPLIVSTVCYTSIIRCLSSSAVANRSKKSRALFLSAAVFCIFIVCFGPTNVLLIVHYLFLSDSPGTEAAYFAYLLCVCVSSVSCCIDPLIYYYASSECQRHLYSILCCKESSDPNSCNSTGQLMPSKMDTCSSHLNNSIYKKLLA

SEQ ID NO: 22 - DNA, 24 bp, adaptor
agcactctccagcctctcaccgca

SEQ ID NO: 23 - DNA, 12 bp, adaptor
gatctgcggtga

SEQ ID NO: 24 - DNA, 24 bp, adaptor
accgacgtcgactatccatgaaca

SEQ ID NO: 25 - DNA, 12 bp, adaptor
gatctgttcatg

SEQ ID NO: 26 - DNA, 24 bp, adaptor
aggcaactgtgctatccgagggaa

SEQ ID NO: 27 - DNA, 12 bp, adaptor
gatcttccctcg

(19) United States
(12) Patent Application Publication
(10) Pub. No.: US 2003/0166150 A1
(43) Pub. Date: Sep. 4, 2003

(54) Asthma associated factors as targets for treating atopic allergies including asthma and related disorders

(75) Inventors: Jean-Christophe Renauld, Krainem (BE); Jamila Louahed, East Norristown, PA (US); Roy Levitt, Ambler, PA (US); Nicholas Nicolaides, Media, PA (US); Qu Dong, Lansdale, PA (US)

Correspondence Address: MORGAN LEWIS & BOCKIUS LLP, 1111 PENNSYLVANIA AVENUE NW, WASHINGTON, DC, 20004, US

(73) Assignee: Ludwig Institute for Cancer Research

(21) Appl. No.: 10/348,190

(22) Filed: Jan. 22, 2003

Related U.S. Application Data
(62) Division of application No. 09/157,249, filed on Sep. 18, 1998, now abandoned.
(60) Provisional application No. 60/059,510, filed on Sep. 19, 1997.

(51) Int. Cl. C12P 21/02; C07K 14/705; C12N 5/06; C07H 21/04

(52) U.S. Cl. 435/69.1; 435/320.1; 435/325; 530/350; 536/23.5

(57) ABSTRACT

A new gene in the G-coupled protein receptor family is discribed that is induced by IL-9, thereby providing a therapeutic target in IL-9 mediated development of atopic allergy, asthma-related disorders and certain lymphomas or leukemias. A method for recombinantly producing the polypeptide encoded by the gene is disclosed. A method for identification and use of agonist and antagonists of GCR9 to treat atopic allergy, asthma-related disorders and certain lymphomas or leukemias is also included. A method for diagnosing susceptibility to, and assessing treatment of atopic allergy, asthma-related disorders and certain lymphomas or leukemias by measuring the level of GCR9 in biological samples using antibody specific for the GCR9 polypeptide or nucleic acid probes specific for GCR9 nucleic acids is also disclosed.

### CROSS-REFERENCE TO RELATED APPLICATION

This application claims the benefit of U.S. Provisional Patent Application No. 60/059,510 which was filed Sep. 19, 1997. This invention is related to the subject matter of U.S. patent application Ser. Nos. 08/697,419; 08/697,360; 08/697,473; 08/697,472; 08/697,471; 08/702,105; 08/702,110; 08/702,168; 08/697,440; and 08/874,503, ail of which were filed on Aug. 23, 1996 and are incorporated herein by reference. This application is also related to U.S. Provisional Patent Application No. 60/032,224 which was filed December 2, 1996 and which is incorporated herein by reference.

### FIELD OF THE INVENTION

This invention relates to modulating activities associated with the IL-9 pathway for the treatment of atopic allergies and related disorders like asthma. It also relates to inhibition of the IL-9 pathway for the treatment of cancer.

### BACKGROUND OF THE INVENTION

Inflammation is a complex process in which the body's defense system combats foreign entities. While the battle against foreign entities may be necessary for the body's survival, some defense systems respond to foreign entities, even innocuous ones, as dangerous and thereby damage surrounding tissue in the ensuing battle.

Atopic allergy, or atopy, is an ecogenetic disorder, where genetic background dictates the response to environmental stimuli, such as pollen, food, dander and insect venoms. The disorder is generally characterized by an increased ability of lymphocytes to produce IgE antibodies in response to ubiquitous antigens. Activation of the immune system by these antigens leads to allergic inflammation and may occur after ingestion, penetration through the skin or after inhalation. When this immune activation occurs and is accompanied by pulmonary inflammation and bronchial hyperresponsiveness, this disorder is broadly characterized as asthma. Certain cells are critical to this inflammatory reaction and they include T cells and antigen-presenting cells, B cells that produce IgE, and basophils and eosinophils that bind IgE. Airway infiltration of eosinophils is a hallmark of asthma which results in damage to airway epithelium. Considerable evidence exists supporting a relationship between eosinophils and bronchial hyperresponsiveness (Devos et al., 1995). Therefore, prevention of eosinophil accumulation in airway epithelium through inhibition of cellular recruitment signaling cascades represents a therapeutic target for potential anti-inflammatory drugs for the treatment of asthma.

While asthma is generally defined as an inflammatory disorder of the airways, clinical symptoms arise from intermittent air flow obstruction. It is a chronic, disabling disorder that appears to be increasing in prevalence and severity (Gergen et al., 1992). It is estimated that 30-40% of the population suffer with atopic allergy and 15% of children and 5% of adults in the population suffer from asthma (Gergen et al, 1992). Thus, an enormous burden is placed on our health-care resources.

Interestingly, while most individuals experience similar environmental exposures, only certain individuals develop atopic allergy and asthma. This hypersensitivity to environmental allergens known as “atopy” is often indicated by elevated serum IgE levels or abnormally intense skin test response to allergens in atopic individuals as compared to nonatopics (Marsh et al., 1982). Strong evidence for a close relationship between atopic allergy and asthma is derived from the fact that most asthmatics have clinical and serologic evidence of atopy (Clifford et al., 1987; Gergen, 1991; Burrows et al., 1992; Johannson et al., 1972; Sears et al., 1991; Halonen et al., 1992). In particular, younger asthmatics have a high incidence of atopy (Marsh et al., 1982). In addition, immunologic factors associated with an increase in total serum IgE levels are very closely related to impaired pulmonary function (Burrows et al., 1989).

Both the diagnosis and treatment of these disorders are problematic (Gergen et al., 1992). The assessment of inflamed lung tissue is often difficult and frequently the source of the inflammation cannot be determined. Without knowledge of the source of the airway inflammation and protection from the inciting foreign environmental agent or agents, the inflammatory process cannot be interrupted. It is now generally accepted that failure to control pulmonary inflammation leads to significant loss of lung function over time.

Current treatments suffer their own set of disadvantages. The main therapeutic agents, β agonists, reduce the symptoms thereby transiently improving pulmonary function, but do not affect the underlying inflammation so that lung tissue remains in jeopardy. In addition, constant use of β agonists results in desensitization which reduces their efficacy and safety (Molinoff et al., 1995). The agents that can diminish the underlying inflammation, the anti-inflammatory steroids, have their own list of disadvantages that range from immunosuppression to bone loss (Molinoff et al., 1995).

Because of the problems associated with conventional therapies, alternative treatment strategies have been evaluated. Glycophorin A (Chu et al., 1992), cyclosporin (Alexander et al., 1992; Morely, 1992) and a nonapeptide fragment of interleukin 2 (IL-2) (Zavyalov et al., 1992) all inhibit potentially critical immune functions associated with homeostasis. What is needed in the art is a treatment for asthma that addresses the underlying pathogenesis. Moreover, these therapies must address the episodic nature of the disorder and the close association with allergy and intervene at a point downstream from critical immune functions.

In the related patent applications mentioned above, it was demonstrated that interleukin 9 (IL-9), its receptor and activities effected by IL-9 are the appropriate targets for therapeutic intervention in atopic allergy including asthma and related disorders. Applicants now disclose related genes that are important in atopic allergy, asthma and certain lymphomas as well as methods of regulating these genes for therapeutic intervention.

Mediator release from mast cells by allergen has long been considered a critical initiating event in allergy. IL-9 was originally identified as a mast cell growth factor (Schmitt et al., 1989) and applicants have previously demonstrated that IL-9 appears to up-regulate the expression of mast cell proteases including MCP-1, MCP-2, MCP-4 (Godfraind et al., 1998) and granzyme B (Louahed et al., 1995). Thus, IL-9 may serve a role in the proliferation and differentiation of mast cells. Moreover, IL-9 up-regulates the expression of the alpha chain of the high affinity IgE receptor (Louahed et al., 1995). Elevated IgE levels are considered to be a hallmark of atopic allergy and a risk factor for asthma. Furthermore, both in vitro and in vivo studies have shown IL-9 to potentiate the release of IgE from primed B cells (Dugas et al., 1993; Petit-Frere et al., 1993).

Based on the data presented in the related patents listed above, there is substantial support for the IL-9 gene candidate in asthma. First, applicants demonstrate linkage homology between humans and mice, suggesting the same gene is responsible for producing biologic variability in response to antigen in both species. Second, differences in expression of the murine IL-9 candidate gene were associated with biologic variability in bronchial responsiveness. In particular, a loss of function is associated with a lower baseline bronchial response in B6 mice. Third, recent evidence for linkage disequilibrium in data from humans suggests IL-9 may be associated with atopy and bronchial hyperresponsiveness consistent with a role for this gene in both species (Doull et al., 1996). Moreover, applicants have demonstrated that a genetic alteration in the human gene appears to be associated with loss of cytokine function and lower IgE levels. Fourth, the pleiotropic functions of this cytokine and its receptor in the allergic immune response strongly support a role for the IL-9 pathway in the complex pathogenesis of asthma. Fifth, in humans, biologic variability in the IL-9 receptor also appears to be associated with atopic allergy and asthma. Finally, despite the inherited loss of IL-9 receptor function, these individuals appear to be otherwise healthy. Thus, nature has demonstrated in atopic individuals that the therapeutic down-regulation of IL-9 and IL-9 receptor genes or genes activated by IL-9 and its receptor is likely to be safe.

Of equal importance to the relationship of IL-9 with atopic disorders is its connection to cell proliferation and differentiation. IL-9 was also initially characterized for its ability to promote growth of T helper cells (Uyttenhove et al., 1988). Subsequently, several other activities were attributed to IL-9 including: differentiation of hematopoietic and neuronal progenitor cells and proliferation as well as differentiation of mast cells (Renauld et al., 1995). In addition, there is some evidence for involvement of IL-9 in both human and murine tumorigenesis (Vink et al., 1993). Overexpression of IL-9 has been associated with a high susceptibility to T cell lymphomas in vivo and an autocrine IL-9 loop has been characterized in some human Hodgkin lymphomas (Renauld et al., 1994; Merz et al., 1991). It therefore seems likely that IL-9 is also involved in other neoplasms of T cell origin including T cell leukemias and Mycosis fungoides. Thus, the art now understands how the IL-9 gene, its receptor and their functions are related to atopic allergy, asthma, cell proliferation, transformation and tumorigenesis. Therefore, a specific need in the art exists for elucidation of the role of genes which are regulated by IL-9 in the etiology of these disorders. Furthermore, most significantly, based on this knowledge, there is a need for the identification of agents that are capable of regulating the activity of these genes or their gene products for treating these disorders.

### SUMMARY OF THE INVENTION

Applicants have identified a new gene from the seven trans-membrane, G protein-coupled receptor family designated GCR9 that is selectively up-regulated by IL-9 and therefore is part of the IL-9 signaling pathway.

In a first embodiment, the invention provides purified and isolated DNA molecules having nucleotide sequences encoding human GCR9 or functionally effective fragments thereof.

The invention further provides purified and isolated protein molecules having amino acid sequences comprising human GCR9 or functionally effective fragments thereof.

In a second embodiment, the invention provides purified and isolated DNA molecules having nucleotide sequences encoding murine GCR9 or functionally effective fragments thereof.

The invention further provides purified and isolated protein molecules having amino acid sequences comprising murine GCR9 or functionally effective fragments thereof.

Functionally effective fragments include, but are not limited to, domains of GCR9 which bind to the cognate ligand and domains which elicit GCR9 dependent physiological signal transduction mechanism(s).

Applicants have satisfied the need for diagnosis and treatment for atopic allergy, asthma and certain lymphomas or leukemias by demonstrating the role of GCR9 in the pathogenesis of these disorders. Therapies for these disorders are derived from the down-regulation of GCR9 as a member of the IL-9 pathway.

The identification of GCR9 has led to the discovery of compounds capable of down-regulating its activity. Molecules that down-regulate GCR9 are therefore claimed in the invention. Down-regulation is defined here as a decrease in activation, function or synthesis of GCR9, its ligands or activators. It is further defined as an increase in degradation of GCR9, its ligands or activators. It is still further defined as the removal of the receptor from the cell surface or uncoupling of the receptor from secondary effectors (e.g., G-proteins, ion channels, nucleotide cyclases, phospholipases, and phosphodiesterases). Down-regulation is therefore achieved in a number of ways. For example, by administration of molecules that can destabilize binding of GCR9 with its ligands. Such molecules encompass polypeptide products, including those encoded by the DNA sequences of the GCR9 gene or DNA sequences containing various mutations of this gene. These mutations may be point mutations, insertions, deletions or spliced variants of the GCR9 gene. This invention also includes truncated polypeptides encoded by the DNA molecules described above. These polypeptides being capable of interfering with the interaction of GCR9 with its ligands and other proteins.

A further embodiment of this invention includes the down-regulation of GCR9 function by altering expression of the GCR9 gene, the use of antisense therapy being an example. Down-regulation of GCR9 expression is accomplished by administering an effective amount of antisense oligonucleotide. These antisense molecules can be fashioned from the DNA sequence of the GCR9 gene or sequences containing various mutations, deletions, insertions or spliced variants. Another embodiment of this invention relates to the use of isolated RNA or DNA sequences derived from the GCR9 gene. These sequences contain various mutations such as point mutations, insertions, deletions or spliced variant mutations of the GCR9 gene and can be useful in gene therapy.

This invention further includes small molecules with the necessary three-dimensional structure required to bind with sufficient affinity to block the interaction of GCR9 with its ligands. GCR9 blockade, resulting in down-regulation of GCR9 activity, calcium flux and other processes of proinflammatory cells where it is expressed, make these molecules useful in treating inflammation associated with atopic allergy, asthma and related disorders. GCR9 blockade by these same molecules make them useful for the treatment of certain lymphomas or leukemias as well.

In a further embodiment, aminosterol compounds are shown to inhibit GCR9 induction by IL-9 or antigen and therefore are useful in treating atopic allergies, asthma and certain lymphomas or leukemias. In yet another embodiment, inhibitors that block activation pathways downstream from GCR9 are shown to down-regulate the IL-9 pathway and therefore can also be used for the treatment of atopic allergies, asthma and certain lymphomas or leukemias.

The products discussed above represent various effective therapeutic agents in treating atopic allergy, asthma and certain lymphomas or leukemias. Applicants have provided antagonists and methods for identifying antagonists that are capable of down-regulating GCR9. Applicants also provide methods for down-regulating GCR9 activity by administering truncated protein products, aminosterols or the like.

Applicants also provide a method for the diagnosis of susceptibility to atopic allergy, asthma and certain lymphomas or leukemias by describing a method for assaying the induction of GCR9, its functions or downstream activities. In a further embodiment, applicants provide methods to monitor the effects of GCR9 down-regulation as a means to follow the treatment of atopic allergy, asthma and certain lymphomas or leukemias.

The accompanying figures, which are incorporated in and constitute a part of this specification, illustrate several embodiments of the invention and, together with the description, serve to explain the principle of the invention.

### BRIEF DESCRIPTION OF FIGURES

FIG. 1: cDNA (SEQ ID NO:1) and deduced amino acid (SEQ ID NO:2) sequence of the murine GCR9 gene.

FIG. 2: Dendrogram of the related members of the G protein-coupled gene family.

FIG. 3: Hydropathy plot of murine GCR9 and alignment of GCR9 to the thrombin receptor.

FIG. 4: Northern blot demonstrating tissue specific distribution of murine GCR9.

FIG. 5: Northern blot demonstrating murine GCR9 induction by IL-9 but not by IL-2 in ST2K9 and TS6 cells in vitro.

FIG. 6: RT-PCR demonstrating GCR9 expression in IL-9 transgenic mice (Tg5) but not the parental strain (FVB).

FIG. 7: RT-PCR demonstrating GCR9 induction by mitogen in splenocytes from C57BL6J.

FIG. 8: RT-PCR demonstrating GCR9 induction by mitogen in splenocytes from DBA2J mice.

FIG. 9: Northern blot demonstrating murine GCR9 induction by IL-9 but not IL-3 from bone marrow derived primary mast cells.

FIG. 10: cDNA (SEQ ID NO:3) and deduced amino acid (SEQ ID NO:4) sequence of the human GCR9 gene.

FIG. 11: Northern blot demonstrating tissue specific distribution of human GCR9.

FIG. 12: RT-PCR demonstrating human GCR9 expression in eosinophils and induction in mast cells.

FIG. 13: Structure of aminosterols tested for inhibition of concanavalin A induced GCR9 expression in murine splenocytes.

FIG. 14: Inhibition of concanavalin A induced GCR9 expression in murine splenocytes by different aminosterols.

FIG. 15: Western blot demonstrating polyclonal antibody sera against murine GCR9.

### DETAILED DESCRIPTION OF THE INVENTION

Applicants have resolved the needs in the art by elucidating a gene in the IL-9 pathway, herein referred to as GCR9, and identifying compositions that affect that pathway which may be used in the diagnosis, prevention or treatment of atopic allergy, asthma and certain lymphomas or leukemias. Asthma encompasses inflammatory disorders of the airways with reversible airflow obstruction. Atopic allergy refers to atopy and related disorders including asthma, bronchial hyperresponsiveness, rhinitis, urticaria, allergic inflammatory disorders of the bowel, and various forms of eczema. Atopy is a hypersensitivity to environmental allergens expressed as the elevation of serum IgE or abnormal skin test responses to allergens as compared to controls. Bronchial hyperresponsiveness is defined here as a heightened bronchoconstrictor response to a variety of stimuli.

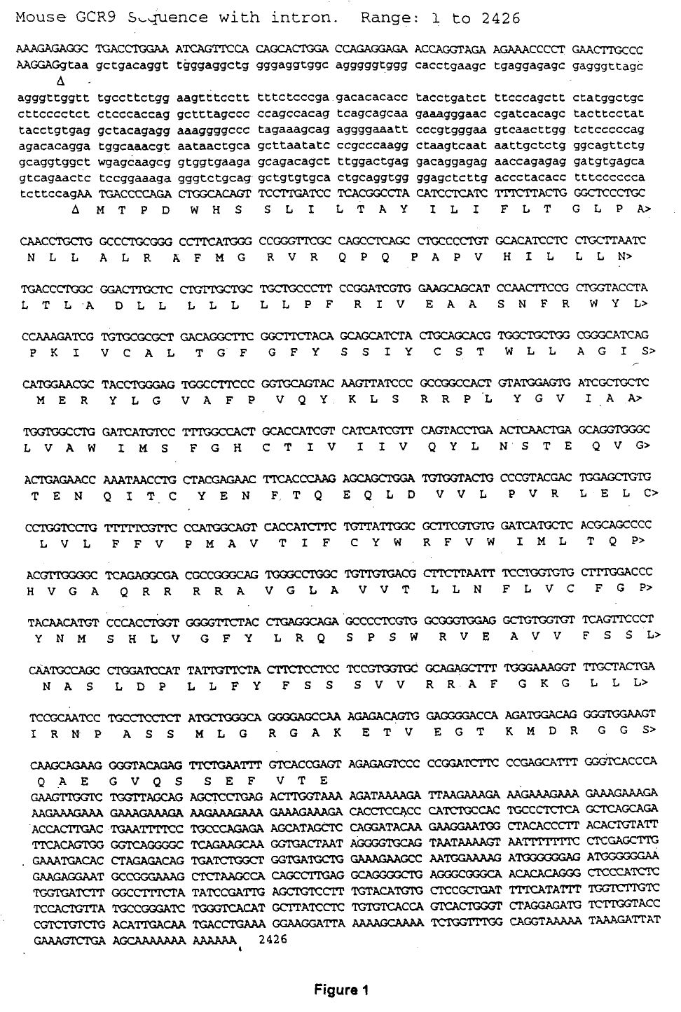

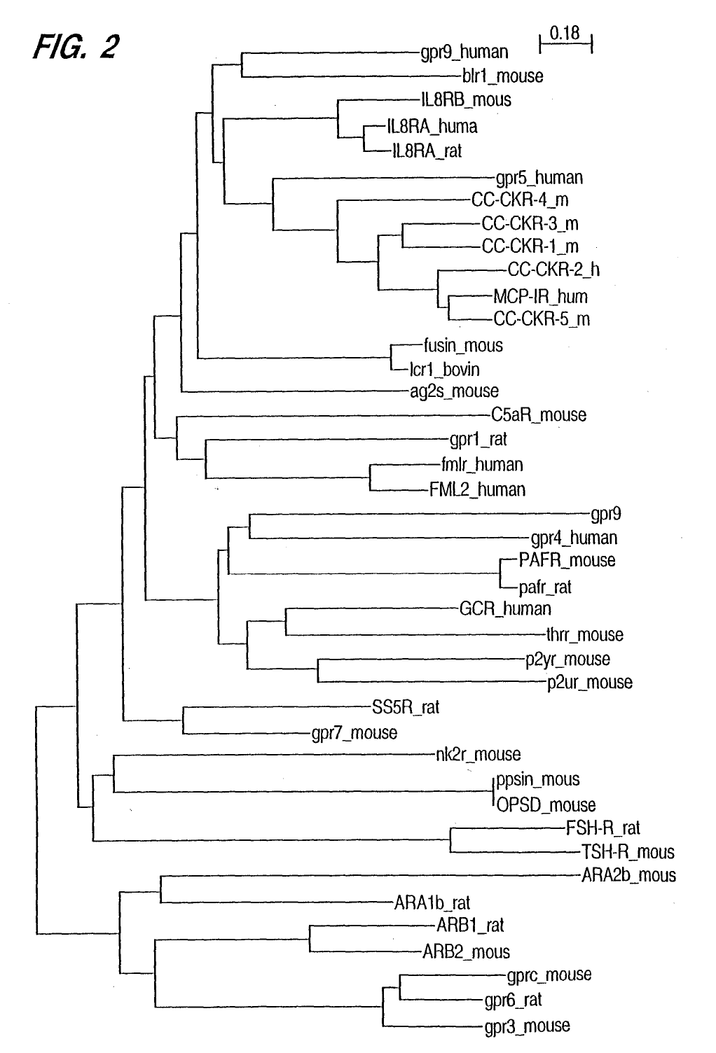

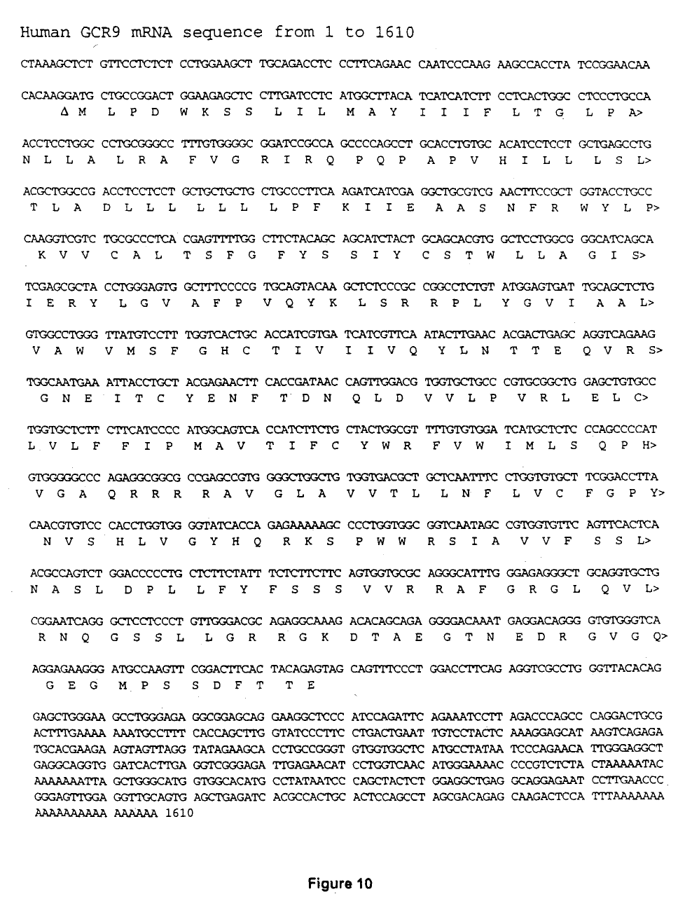

The murine and human cDNA for GCR9 have been isolated. The murine (FIG. 1) and human (FIG. 10) GCR9 genes both encode a 330 amino acid protein. The murine GCR9 amino acid sequence exhibits >80% identity with the human sequence. Analysis of the structure of murine GCR9 indicates significant homology with many G protein-coupled receptors (FIG. 2). Sequence analysis indicated that GCR9 shares the highest homology with the purinergic receptor family. The largest extracellular domain of the human and murine GCR9 displayed significant homology with several G protein-coupled orphan receptors on chromosome 19q13.3 (Sawzdargo et al., 1997) and a IL-2 induced G protein-coupled receptor in T cells derived from chicken (Kaplan et al., 1993). Other more distally related members of the G protein-coupled receptor family which display homology at the nucleotide and amino acid level include thrombin receptors and platelet activating factor.

The human GCR9 gene was identified on the long arm of chromosome 19, which is syntenic with mouse chromosome seven, by hybrid cell mapping. Both murine and human GCR9 genes contain an 1.2 kilobase intron upstream of the start codon as identified by Δ in FIGS. 1 & 10. The nucleotide sequence of the human GCR9 gene displayed some homology with those of other genes in close proximity on chromosome 19, particularly with GPR40 (44%), GPR41 (54%) and GPR42 (54%). The role of GCR9 in atopic disease is further supported by genetic linkage studies that describe a locus near chromosome 19q13 which is important in asthma (The Collaborative Study on the Genetics of Asthma—A Genome wide Search for Asthma Susceptibility Loci in Ethnically Diverse Populations, 1997). Thus, biologic variability in a nearby gene must contribute to the asthmatic response.

Accordingly, the invention provides a purified and isolated nucleic acid molecule comprising a nucleotide sequence encoding murine (FIG. 1) or human (FIG. 10) GCR9, or a fragment thereof. The invention also includes degenerate sequences of the DNA as well as sequences that are substantially homologous. The exemplified source of the GCR9 of the invention is murine and human, although GCR9 from any source is encompassed by the invention. The nucleic acid molecule or fragments thereof may be synthesized using methods known in the art. It is also possible to produce the compound by genetic engineering techniques, by constructing DNA by any accepted technique, cloning the DNA in an expression vehicle and transfecting the vehicle into a cell which will express the compound. (see Sambrook et al., Molecular Cloning—A Laboratory Manual, 2nd edition, Cold Spring Harbor Laboratory Press, 1985).

Nucleic acid molecules of the invention include polynucleotides encoding murine and human GCR9 with the sequences of SEQ ID NO:1 and SEQ ID NO:3, respectively, as well as all nucleic acid sequences complementary to these sequences. A complementary sequence may include an antisense nucleotide.

It is understood that all polynucleotides encoding all or a portion of GCR9 are also included herein, as long as they encode a polypeptide with the functional activities of GCR9 as set forth herein. Polynucletide sequences of the invention include DNA, cDNA, synthetic DNA and RNA sequences which encode GCR9. Such polynucleotides also include naturally occurring, synthetic and intentionally manipulated polynucleotides. For example, such polynucleotide sequences may comprise genomic DNA which may or may not include naturally occurring introns. Moreover, such genomic DNA may be obtained in association with promoter regions or polyadenylated sequences. As another example, portions of the mRNA sequence may be altered due to alternate RNA splicing patterns or the use of alternate promoters for RNA transcription. As yet another example, GCR9 polynucleotides may be subjected to site-directed mutagenesis.

The polynucleotides of the invention further include sequences that are degenerate as a result of the genetic code. The genetic code is said to be degenerate because more than one nucleotide triplet codes for the same amino acid. There are twenty natural amino acids, most of which are specified by more than one codon. It will be appreciated by those skilled in the art that as a result of the degeneracy of the genetic code, a multitude of nucleotide sequences, some bearing minimal nucleotide sequence homology to the nucleotide sequences of SEQ ID NO:1 and SEQ ID NO:3 may be produced as a result of this invention. Therefore, all degenerate nucleotide sequences are included in the invention as long as the amino acid sequence of the GCR9 polypeptide encoded by the nucleotide sequence is functionally unchanged or substantially similar in function. The invention specifically contemplated each and every possible variation of peptide or nucleotide sequence that could be made by selecting combinations based on the possible amino acid and codon choices made in accordance with the standard triplet genetic code as applied to the sequences of SEQ ID NO:1 and SEQ ID NO:3 and all such variations are to be considered specifically disclosed herein.

Also included in the invention are fragments (portions, segments) of the sequences disclosed herein which selectively hybridize to the sequences of SEQ ID NO:1 and SEQ ID NO:3. Selective hybridization as used herein refers to hybridization under stringent conditions (see Maniatis et al., Molecular Cloning—A Laboratory Manual, Cold Spring Harbor Laboratory Press, 1989), which distinguishes related from unrelated nucleotide sequences. The active fragments of the invention, which are complementary to mRNA and the coding strand of DNA, are usually at least about fifteen nucleotides, more usually at least twenty nucleotides, preferably thirty nucleotides and more preferably may be fifty nucleotides or more.

As used herein, “stringent conditions” are conditions in which hybridization yields a clear and readable sequence. Stringent conditions are those that (1) employ low ionic strength and high temperature for washing, for example; 0.015 M sodium chloride, 0.0015 M sodium citrate, 0.1% SDS buffer at 50° C., or (2) employ during hybridization a denaturing agent such as formamide, for example; 50% formamide with 0.1% bovine serum albumin, 0.1% Ficoll, 0.1% polyvinylpyrrolidone, 50 mM sodium phosphate buffer at pH 6.5 with 750 mM sodium chloride, 75 mM sodium citrate at 42° C. Another example is using 50% formamide, 5×SSC, 50 mM sodium phosphate (pH 6.8), 0.1% sodium pyrophosphate, 5×Denhardt's solution, sonicated salmon sperm DNA (50 μg/ml), 0.1% SDS and 10% dextran sulfate at 42° C., with washes at 42° C. in 0.2×SSC and 0.1% SDS. A skilled artisan can readily determine and vary the stringency conditions appropriately to obtain a clear and detectable hybridization signal.

The present invention provides nucleic acid molecules encoding GCR9 proteins which hybridize with nucleic acid molecules comprising sequences complementary to either SEQ ID NO:1 or SEQ ID NO:3 under conditions of sufficient stringency to produce a clear signal. As used herein, “nucleic acid” is defined as RNA or DNA encoding GCR9 peptides, or complementary to nucleic acids encoding such peptides, or hybridize to such nucleic acids and remain stably bound to them under stringent conditions, or encode polypeptides sharing at least 60% sequence identity, preferably at least 75% sequence identity, and more preferably at least 80% sequence identity with the GCR9 peptide sequences. Nucleic acids of the invention also include nucleic acid molecules which comprise nucleotide sequences sharing at least 60% or 70% sequence identity with the open-reading-frame of SEQ ID NO:1 or to SEQ ID NO:3, preferably 80% or 85% sequence identity with the open-reading-frame of SEQ ID NO:1 or to SEQ ID NO:3, or more preferably, 90%, 91%, 95% or 97% sequence identity with the open-reading-frame of SEQ ID NO:1 or to SEQ ID NO:3.

Homology or identity is determined by BLAST (Basic Local Alignment Search Tool) analysis using the algorithm employed by the programs blastp, blastn, blastx, tblastn and tblastx (Karlin et al, Proc. Natl. Acad. Sci. USA 87: 2264-2268 (1990) and Altschul, S. F. J. Mol. Evol. 36: 290-300(1993), fully incorporated by reference) which are tailored for sequence similarity searching. The approach used by the BLAST program is to first consider similar segments between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al. (Nature Genetics 6: 119-129 (1994)) which is fully incorporated by reference. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff, et al. Proc. Natl. Acad. Sci. USA 89: 10915-10919 (1992), fully incorporated by reference). For blastn, the scoring matrix is set by the ratios of M (i.e., the reward score for a pair of matching residues) to N (i.e., the penalty score for mismatching residues), wherein the default values for M and N are 5 and −4, respectively.

The invention further provides substantially pure GCR9 polypeptides. The term “substantially pure” as used herein refers to GCR9 polypeptide which is substantially free of other proteins, lipids, carbohydrates or other materials with which it is naturally associated. One skilled in the art can purify GCR9 using standard techniques for protein purification.

The invention further provides for the production of GCR9 polypeptides recombinantly. In a related aspect, the DNA encoding GCR9, including variations and substitutions of the native sequence, is operably linked to a cloning vehicle, wherein said vehicle is a plasmid, cosmid, artificial chromosome or viral vector or a combination of said vehicles (see Kreigler, 1990). The DNA in operable linkage may comprise a bacterial promoter or eukarotic promoter and/or enhancer elements for expression in prokaryotic and/or eukaryotic host cells (see Kreigler, in Gene Transfer and Expression: A Laboratory Manual 1990, M Stockton Press, New York and Sambrook, et al., in Molecular Cloning: A Laboratory Manual, 1989, Cold Spring Harbor Laboratory Press, Cold Spring, New York). Host cells transfected or transformed with cloning vehicles comprising DNA encoding GCR9 in operable linkage can be used to produce the polypeptide, wherein the cells are grown under suitable conditions to express GCR9 and wherein the protein is separated from the host cells and surrounding nutrients (including purification from inclusion bodies, see Sambrook, et al., 1989). In a related aspect, transcriptional elements are defined as sequences of nucleic acids which direct RNA polymerases such that expression of mRNA is regulated. The invention also provides amino acid sequences coding for murine GCR9 polypeptides (SEQ ID NO:2) and human GCR9 polypeptides (SEQ ID NO:4). The polypeptides of the invention include those which differ from SEQ ID NO:2 and SEQ ID NO:4 as a result of conservative variations. The terms “conservative variation” or “conservative substitution” as used herein denotes the replacement of an amino acid residue by another, biologically similar residue. Conservative variations or substitutions are not likely to change the shape of the polypeptide chain. Examples of conservative variations, or substitutions, include the replacement of one hydrophobic residue such as isoleucine, valine, leucine or methionine for another, or the substitution of one polar residue for another, such as the substitution of arginine for lysine, glutamic for aspartic acid, or glutamine for asparagine, and the like. Therefore, all conservative substitutions are included in the invention as long as the GCR9 polypeptide encoded by the nucleotide sequence is functionally unchanged or similar.

As used herein, an isolated GCR9 protein can be a full-length GCR9 protein or any homologue of such a protein, such as a GCR9 protein in which amino acids have been deleted (for example, a truncated version of the protein, such as a peptide), inserted, inverted, substituted and derivatized (for example, by glycosylation, phosphorylation, acetylation, myristoylation, prenylation, palmitoylation, amidation and addition of glycosylphosphatidyl inositol), wherein modified protein retains the physiological characteristics of natural GCR9. A homologue of a GCR9 protein is a protein having an amino acid sequence that is sufficiently similar to a natural GCR9 protein amino acid sequence that a nucleic acid sequence encoding the homologue is capable of hybridizing under stringent conditions to a nucleic acid sequence encoding the natural GCR9 protein amino acid sequence. Appropriate stringency requirements are discussed above.

GCR9 protein homologues can be the result of allelic variation of a natural gene encoding a GCR9 protein. A natural gene refers to the form of the gene found most often in nature. GCR9 protein homologues can be produced using techniques known in the art including, but not limited to, direct modifications to a gene encoding a protein using, for example, classic or recombinant DNA techniques to effect random or targeted mutagenesis.

Minor modifications of the GCR9 primary amino acid sequence may result in proteins which have substantially equivalent activity as compared to the GCR9 polypeptides described herein in SEQ ID NO:2 and SEQ ID NO:4. As used herein, a “functional equivalent” of the GCR9 protein is a protein which possesses a biological activity or immunological characteristic substantially similar to a biological activity or immunological characteristic which is peculiar to non-recombinant, or natural, GCR9. The term “functional equivalent” is intended to include the fragments, variants, analogues, homologues, or chemical derivatives of a molecule which possess the biological activity of the GCR9 proteins of the present invention. The biological activity of GCR9 maybe defined as the stimulation of signaling properties observed when cells which do not make GCR9 physiologically are transfected to express recombinant GCR9 and are compared to untransfected cells, wherein there is only stimulation of second messengers (e.g., cAMP, cGMP, Ca²⁺, Pl) in transfected cells when each set of cells is contacted with a compound (see Ryan, et al. J. Biol. Chem. (1998) Vol. 273(22): 13613-13624 and Stables, et al. Anal. Biochem. (1997) Vol252(1): 115-126, for assessing physiological signal transduction mechanisms for orphan receptors, both fully incorporated by reference).

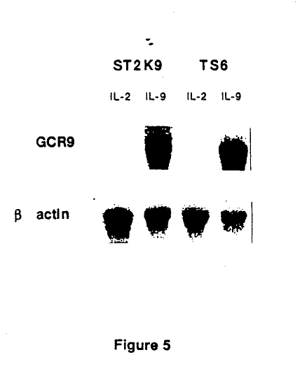

A murine GCR9 probe readily detected abundant amounts of a 1.8 kilobase mRNA in mouse cytokine dependent T helper cell lines TS6 and ST2K9 when cultured in the presence of IL-9 but not IL-2 (FIG. 5). The induction of this gene by concanavalin A in murine splenocytes suggests that this gene plays a role in mitogen signaling. Furthermore, this gene product is specifically induced in the lung and intestine in vivo by the over expression of IL-9 in transgenic animals (Godfraind et al., 1998). This data further confirms that GCR9 is specifically induced by IL-9 and plays a role in the these organs, especially where biologic variability in structure or function of IL-9 and its receptor assume a role in the pathophysiology of atopic allergy including asthma and related disorders.

Evidence defining the role of GCR9 in the pathogenesis of atopic allergy including bronchial hyperresponsiveness, asthma and related disorders derives directly from the applicants' observation that IL-9 selectively induces GCR9. Thus, the pleiotropic role for IL-9 which is critical to a number of antigen induced responses is, in part, dependent on the regulation of GCR9 which plays a role in the physiology of a number of cells critical to atopic allergy. When the functions of IL-9 are down-regulated by antibody pretreatment prior to aerosol challenge with antigen, the animals can be completely protected from the antigen induced responses. These responses include bronchial hyperresponsiveness, eosinophilia and elevated cell counts in bronchial lavage, histologic changes in lung associated with inflammation and elevated total serum IgE. Thus, the treatment of such responses by down-regulating GCR9, which are critical to the pathogenesis of atopic allergy and which characterize the allergic inflammation associated with atopic allergy, are within the scope of this invention.

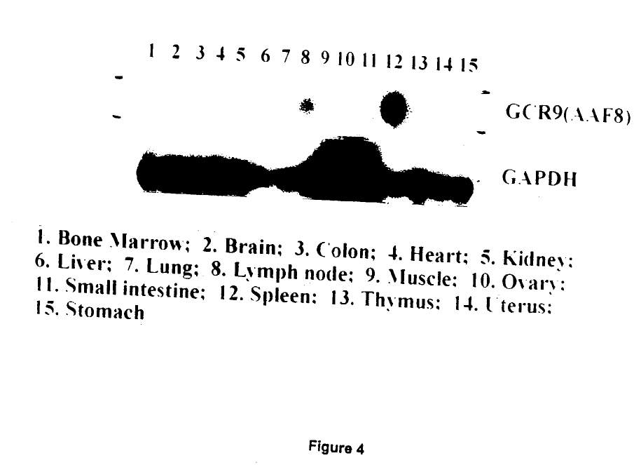

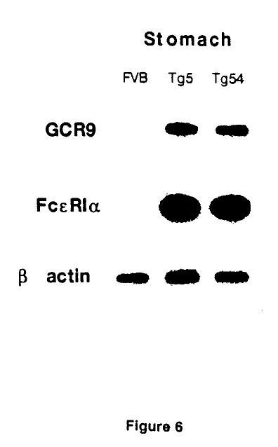

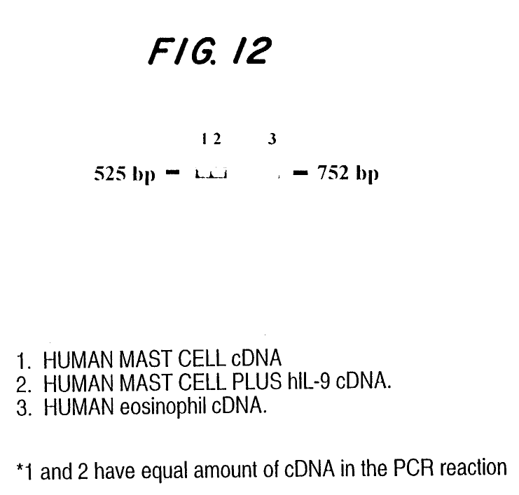

Applicants have shown that GCR9 is induced in cells closely associated with the asthmatic response. For example, murine GCR9 expression is induced in IL-9 stimulated T cells (FIG. 5) and in the stomach of IL-9 transgenic mice (FIG. 6). Tissue distribution studies demonstrate that GCR9 is expressed in many other tissues including lymphoreticular tissue (FIG. 4) where it is likely to be induced in cell types that are important in asthma such as eosinophils or B cells. Human GCR9 is expressed in eosinophils and mast cells and induction occurs following exposure to IL-9 (FIG. 12). This induction in eosinophils establishes a role for GCR9 in both the inflammatory response and asthma as eosinophilia is a consistent feature associated with these processes (Gleich, 1990). Therapies for the treatment of asthma are therefore derived from blockade inflammatory response through inhibition cellular signaling cascade through blockade of GCR9 on eosinophils and mast cells.

Applicant teaches methods of diagnosing susceptibility to atopic allergy and related disorders and for treating these disorders based on the relationship between IL-9, its receptor and GCR9. One diagnostic embodiment involves the recognition of variations in the DNA sequence of GCR9. One method involves the introduction of a nucleic acid molecule (also known as a probe) having a sequence complementary to the GCR9 of the invention under sufficient hybridizing conditions, as would be understood by those in the art. In one embodiment, the sequence will bind specifically to one allele of GCR9, or a fragment thereof, and in another embodiment will bind to multiple alleles. Another method of recognizing DNA sequence variation associated with these disorders is direct DNA sequence analysis by multiple methods well known in the art (Ott, 1991). Another embodiment involves the detection of DNA sequence variation in the GCR9 gene associated with these disorders (Schwengel et al., 1993; Sheffield et al., 1993; Orita et al., 1989; Sarkar et al., 1992; Cotton, 1989). These include the polymerase chain reaction, restriction fragment length polymorphism (RFLP) analysis and single stranded conformational analysis.

In another diagnostic embodiment, susceptibility to asthma-related disorders and certain lymphomas and leukemias associated with elevated levels of GCR9 polypeptide in a human subject can be measured by the steps of: (a) measuring the level of GCR9 polypeptide in a biological sample from said human subject; and (b) comparing the level of GCR9 polypeptide present in normal subjects, wherein an increase in the level of GCR9 polypeptide as compared to normal levels indicates a predisposition to asthma-related disorders and certain lymphomas or leukemias. Such lymphomas and leukemias include chronic lymphocytic leukemia, large granular lymphocyte leukemia, adult diffuse aggressive lymphoma, peripheral T-cell lymphoma, adult T-cell leukemia, acute lymphocytic leukemia and lymphoblastic lymphomas.

In another diagnostic embodiment, therapeutic treatment of asthma or certain lymphomas or leukemias associated with elevated levels of GCR9 polypeptide in a human subject may be monitored by measuring the levels of GCR9 polypeptide in a series of biologic samples obtained at different time points from said subject undergoing therapeutic treatment wherein a significant decrease in said levels of GCR9 polypeptide indicates a successful therapeutic treatment.

Diagnostic probes useful in such assays of the invention include antibodies to GCR9. The antibodies to GCR9 may be either monoclonal or polyclonal, produced using standard techniques well known in the art (see Harlow & Lane's Antibodies: A Laboratory Manual, Cold Spring Harbor Laboratory Press, 1988). They can be used to detect GCR9 by binding to the protein and subsequent detection of the antibody-protein complex by ELISA, Western blot or the like. The GCR9 used to elicit these antibodies can be any of the GCR9 variants discussed above. Antibodies are also produced from peptide sequences of GCR9 using standard techniques in the art (see Protocols in Immunology, John Wiley & Sons, 1994). The peptide sequences from the murine GCR9 that can be used to produce blocking antisera are those which encompass the extracellular portions of the protein and have been identified as MTPDWHSS (residues 1-8) (SEQ ID NO:5); RIVEAASNFRWYLPKIVC (residues 65-82) (SEQ ID NO:6); QYLNSTEQVGTENQITC (residues 148-164) (SEQ ID NO:7); QVGTENQITCYENFTQEQLD (residues 154-174) (SEQ ID NO:8); CYENFTQEQLDWLPVRLE (residues 164-182) (SEQ ID NO:9) and SHLVGFYLRQSPSWR (residues 241-255) (SEQ ID NO:10). Two peptides that have been identified from the extracellular sequence of human GCR9 that will be useful for the production of blocking antisera are KIIEAASNFRWYLPKVVC (residues 65-82) (SEQ ID NO:11) and QYLNTTEQVRSGNEITC (residues 148-164) (SEQ ID NO:12). In addition the human sequences that correspond to murine residues in SEQ ID NO:5 & 8-10 will also be useful for the production of therapeutic antibodies. Fragments of the monoclonals or the polyclonal antisera which contain the immunologically significant portion can also be prepared. Use of immunologically reactive fragments, such as the Fab, Fab′, of F(ab′)₂ fragments is often preferable, especially in a therapeutic context, as these fragments are generally less immunogenic than the whole immunoglobulin.

Assays to detect or measure GCR9 polypeptide in a biological sample with an antibody probe may be based on any available format. For instance, in immunoassays where GCR9 polypeptides are the analyte, the test sample, typically a biological sample, is incubated with anti-GCR9 antibodies under conditions that allow the formation of antigen-antibody complexes. Various formats can be employed, such as “sandwich” assay where antibody bound to a solid support is incubated with the test sample; washed, incubated with a second, labeled antibody to the analyte; and the support is washed again. Analyte is detected by determining if the second antibody is bound to the support. In a competitive format, which can be either heterogeneous or homogeneous, a test sample is usually incubated with an antibody and a labeled competing antigen, either sequentially or simultaneously. These and other formats are well known in the art.

A further embodiment of the invention relates to antisense or gene therapy. It is now known in the art that altered DNA molecules can be tailored to provide a specific selected effect, when provided as antisense or gene therapy. The native DNA segment coding for GCR9 has, as do all other mammalian DNA strands, two strands; a sense strand and an antisense strand held together by hydrogen bonds. The mRNA coding for GCR9 has a nucleotide sequence identical to the sense strand, with the expected substitution of thymidine by uridine. Thus, based upon the knowledge of the GCR9 sequence, synthetic oligonucleotides can be synthesized. These oligonucleotides can bind to the DNA and RNA coding for GCR9. The active fragments of the invention, which are complementary to mRNA and the coding strand of DNA, are usually at least about fifteen nucleotides, more usually at least twenty nucleotides, preferably thirty nucleotides and more preferably may be fifty nucleotides or more. There is no upper limit, other than a practical limit, on the maximal size of such a nucleic acid molecule in that the nucleic acid molecule can include a portion of a gene, an entire gene, or multiple genes, or portions thereof. The binding strength between the sense and antisense strands is dependent upon the total hydrogen bonds. Therefore, based upon the total number of bases in the mRNA, the optimal length of the oligonucleotide sequence may be easily calculated by the skilled artisan. The sequence may be complementary to any portion of the sequence of the mRNA. For example, it may be proximal to the 5′-terminus or capping site or downstream from the capping site, between the capping site and the initiation codon and may cover all or only a portion of the non-coding region or the coding region. The particular site(s) to which the antisense sequence binds will vary depending upon the degree of inhibition desired, the uniqueness of the sequence, the stability of the antisense sequence, etc.

In the practice of the invention, expression of GCR9 is down-regulated by administering an effective amount of synthetic antisense oligonucleotide sequences described above. The oligonucleotide compounds of the invention bind to the mRNA coding for human GCR9 thereby inhibiting expression (translation) of these proteins. The isolated DNA sequences containing various mutations such as point mutations, insertions, deletions or spliced mutations of GCR9 are useful in gene therapy as well.

Antisense oligonucleotides can also be used as tools in vitro to determine the biological function of genes and proteins. Oligonucleotide phosphorothioates (PS-oligos) have also shown great therapeutic potential as antisense-mediated inhibitors of gene expression (Stein et al., 1993 and references therein). Various methods have been developed for the synthesis of antisense oligonucleotides (see Agrawal et al., Methods of Molecular Biology: Protocols for Oligonucleotides and Analogs, Humana Press, 1993 and Eckstein et al., Oligonucleotides and Analogues: A Practical Approach, Oxford University Press, 1991).

In the practice of the invention, expression of GCR9 is modulated by administering an effective amount of synthetic antisense oligonucleotide sequences described above. The oligonucleotide compounds of the invention bind to the mRNA coding for human GCR9 thereby inhibiting expression (translation) of these proteins. The isolated DNA sequences containing various mutations such as point mutations, insertions, deletions, or spliced mutations of GCR9 are useful in gene therapy as well.

The present invention also includes antagonists of GCR9 that block activation of this protein. Antagonists are compounds that are themselves devoid of pharmacological activity but cause effects by preventing the action of an agonist. To identify an antagonist of the invention, one may test for competitive binding with natural ligands of GCR9. Assays of antagonistic binding and activity can be derived from monitoring GCR9 functions for down-regulation as described herein and in the cited literature. One may test for binding to GCR9 to identify allosteric ligands or inverse agonists of the invention. The binding of antagonist may involve all known types of interactions including ionic forces, hydrogen bonding, hydrophobic interactions, van der Waals forces and covalent bonds. In many cases, bonds of multiple types are important in the interaction of an antagonist with a molecule like GCR9.

In a further embodiment, these compounds may be analogues of GCR9 or its ligands. GCR9 analogues may be produced by point mutations in the isolated DNA sequence for the gene, nucleotide substitutions and/or deletions which can be created by methods that are all well described in the art (Simoncsits et al., 1994). This invention also includes spliced variants of GCR9 including isolated nucleic acid sequences of GCR9, which contain deletions of one or more of its exons. The term “spliced variants” as used herein denotes a purified and isolated DNA molecule encoding human GCR9 comprising at least one exon. In addition, these exons may contain various point mutations.

Structure-activity relationships may be used to modify the antagonists of the invention. For example, the techniques of X-ray crystallography and NMR may be used to make modifications of the invention. For example, one can create a three dimensional structure of human GCR9 that can be used as a template for building structural models of deletion mutants using molecular graphics. These models can then be used to identify and construct a ligand for GCR9 with affinity comparable to the natural ligand, but with lower GCR9 activity when compared to the natural ligands. What is meant by lower biologic activity is 2 to 100,000 fold less GCR9 activity than produced by natural ligands, preferably 100 to 1,000 fold less GCR9 activity than produced by natural ligands. In still another embodiment, these compounds may also be used as dynamic probes for GCR9 structure and to develop GCR9 antagonists using cell lines or other suitable means of assaying GCR9 activity.

In addition, this invention also provides compounds that prevent the synthesis or reduce the biologic stability of GCR9. Biologic stability is a measure of the time between the synthesis of the molecule and its degradation. For example, the stability of a protein, peptide or peptide mimetic (Kauvar, 1996) therapeutic may be shortened by altering its sequence to make it more susceptible to enzymatic degradation.

In addition to the direct regulation of the GCR9 gene, this invention also encompasses methods of inhibition of intracellular signaling by GCR9. It is known in the art that highly exergonic phosphoryl-transfer reactions are catalyzed by various enzymes known as kinases. In other words, a kinase transfers phosphoryl groups between ATP and a metabolite. Included within the scope of this invention are specific inhibitors of protein kinases. Thus, inhibitors of these kinases are useful in the modulation of GCR9 and are useful in the treatment of atopic allergies and asthma. Another aspect of GCR9 signaling involves the interaction of GCR9 with a G-protein. Antagonist can be developed which block the GCR9-G protein interaction or which prevent the G protein activation. Such inhibitors of G protein activation include inhibitors of farnesylation, palmytoylation, myristoylation, isoprenylation or geranylgeranylation, all well known in the art. A further aspect of the invention involves the use of inhibitors of other downstream effectors of GCR9 mediated signal transduction. These include inhibitors of Phospholipase C, Phospholipase A2, Adenylyl Cyclase, MAP kinase and Ras which are known downstream effectors for this family of receptors. Another method to antagonize the activity of GCR9 is the use of a ligand for another G protein-coupled receptor which caused heterologous desensitization of GCR9 signal transduction. Examples of this effect are well known in the art.

In still another aspect of the invention, surprisingly, aminosterol compounds were found to be useful in the inhibition of GCR9 induction by mitogen stimulation. Aminosterol compounds which are useful in this invention are described in U.S. patent application Ser. No. 08/290,826 and its related application Ser. Nos. 08/416,883 and 08/478,763 as well as in Ser. No. 08/483,059 and its related application Ser. Nos. 08/483,057, 08/479,455, 08/479,457, 08/475,572, 08/476,855, 08/474,799 and 08/487,443, which are specifically incorporated herein by reference.

In addition, the invention includes pharmaceutical compositions comprising the compounds of the invention together with a pharmaceutically acceptable carrier. Pharmaceutically acceptable carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical carriers are described in Remington's Pharmaceutical Sciences, Mack Publishing Company, Easton Pa., 1995, specifically incorporated herein by reference.

The compounds used in the method of treatment of this invention may be administered systemically or topically, depending on such considerations as the condition to be treated, need for site-specific treatment, quantity of drug to be administered, and similar considerations.

Topical administration may be used. Any common topical formation such as a solution, suspension, gel, ointment or salve and the like may be employed. Preparation of such topical formulations as are well described in the art of pharmaceutical formulations as exemplified, for example, by Remington's Pharmaceutical Sciences. For topical application, these compounds could also be administered as a powder or spray, particularly in aerosol form. The active ingredient may be administered in pharmaceutical compositions adapted for systemic administration. As is known, if a drug is to be administered systemically, it may be confected as a powder, pill, tablets or the like, or as a syrup or elixir for oral administration. For intravenous, intraperitoneal or intra-lesional administration, the compound will be prepared as a solution or suspension capable of being administered by injection. In certain cases, it may be useful to formulate these compounds in suppository form or as an extended release formulation for deposit under the skin or intermuscular injection. In a preferred embodiment, the compounds of this invention may be administered by inhalation. For inhalation therapy the compound may be in a solution useful for administration by metered dose inhalers, or in a form suitable for a dry powder inhaler.

An effective amount is that amount which will modulate GCR9. A given effective amount will vary from condition to condition and in certain instances may vary with the severity of the condition being treated and the patient's susceptibility to treatment. Accordingly, a given effective amount will be best determined at the time and place through routine experimentation. However, it is anticipated that in the treatment of atopic allergy and asthma-related disorders in accordance with the present invention, a formulation containing between 0.001 and 5 percent by weight, preferably about 0.01 to 1 percent, will usually constitute a therapeutically effective amount. When administered systemically, an amount between 0.01 and 100 mg per kg body weight per day, but preferably about 0.1 to 10 mg per kg, will effect a therapeutic result in most instances.

Applicant also provides for a method to screen for the compounds that modulate GCR9 or the functions controlled by GCR9. One may determine whether the functions expressed by GCR9 are down-regulated using techniques standard in the art. In a specific embodiment, applicant provides for a method of identifying compounds which modulate GCR9 in vitro and in vivo. In one embodiment, serum IgE may be measured using techniques well known in the art to assess the efficacy of a compound in down regulating the functions of GCR9 induced by antigen in vivo. In another embodiment, bronchial hyperresponsiveness, bronchoalveolar lavage, and eosinophilia may be measured using techniques well known in the art to assess the efficacy of a compound in down regulating the functions of GCR9 in vivo. In yet another embodiment, the functions of GCR9 may be assessed in vitro. As is known to those in the art, as a G protein-coupled receptor, human GCR9 specifically activates G proteins and these cellular processes can be monitored as a means of assaying functional inhibitors of GCR9.

The invention also includes a simple screening assay for GCR9 binding to in vitro translated signaling proteins. In one embodiment, GCR9 induction can be monitored in vitro and in vivo by isolating cellular RNA and protein and assaying by Western blotting (protein) or RT-PCR (cDNA) the relative amounts of GCR9 that are seen at steady state. In another embodiment the GCR9 promoter can be subcloned in front of a reporter gene, such as luciferase of CAT, and this construct used to identify molecules that block GCR9 induction by measuring the activity of the reporter gene. Such methods are a preferred embodiment of this invention.

The present invention also provides transgenic animals that over-express GCR9 or express GCR9 at a level much lower than that of a wild-type organism. A “wild type” organism is one that is the most frequently observed phenotype for GCR9 expression, usually arbitrarily designated as a “normal” individual.

Transgenic animals are genetically modified animals into which cloned genetic material has been transferred. The cloned genetic material is often referred to as a transgene. The transgene may consist of nucleic acid sequences derived from the genome of the same species or of a different species, including non-animal species, than the species of the target animal.

The development of transgenic technology allows investigators to create mammals of virtually any genotype and to assess the consequences of introducing specific foreign nucleic acid sequences on the physiological and morphological characteristics of the transformed animals. The availability of transgenic animals permits cellular processes to be influenced and examined in a systematic and specific manner not achievable with most other test systems. For example, the development of transgenic animals provides biological and medical scientists with models that are useful in the study of disease. Such animals are also useful for the testing and development of new pharmaceutically active substances. Gene therapy can be used to ameliorate or cure the symptoms of genetically-based diseases.

Transgenic animals can be produced by a variety of different methods including transfection, electroporation, microinjection, biolistics (also called gene particle acceleration or microprojectile bombardment), gene targeting in embryonic stem cells and recombinant viral and retro viral infection (see U.S. Pat. No. 4,736,866; U.S. Pat. No. 5,602,307; Mullins et al., 1993; Brenin et al, 1997; Tuan, Recombinant Gene Expression Protocols. Methods in Molecular Biology, Humana Press, 1997.

The term “knock-out” generally refers to mutant organisms which contain a null allele of a specific gene. The term “knock-in” generally refers to mutant organisms into which a gene has been inserted through homologous recombination. The knock-in gene may be a mutant form of a gene which replaces the endogenous, wild-type gene. Mice which are knock-in or knock-out mice as regards the GCR9 gene are encompassed by the disclosure of this invention.

A number of recombinant rodents have been produced, including those which express an activated oncogene sequence (U.S. Pat. No. 4,736,866); express simian SV 40 T-antigen (U.S. Pat. No. 5,728,915); lack the expression of interferon regulatory factor-i (U.S. Pat. No. 5,731,490); exhibit dopaminergic dysfunction (U.S. Pat. No. 5,723,719); express at least one human gene which participates in blood pressure control (U.S. Pat. No. 5,731,489); display greater similarity to the conditions existing in naturally occurring Alzheimer's disease (U.S. Pat. No. 5,720,936); have a reduced capacity to mediate cellular adhesion (U.S. Pat. No. 5,602,307); and also possess a bovine growth hormone gene (Clutter et al., 1996).

While rodents, especially mice and rats, remain the animals of choice for most transgenic experimentation, in some instances it is preferable or even necessary to use alternative animal species. Transgenic procedures have been successfully utilized in a variety of non-murine animals, including sheep, goats, pigs, dogs, cats, monkeys, chimpanzees, hamsters, rabbits, cows and guinea pigs (see Kim et al., 1997; Houdebine, 1995; Petters, 1994; Schnieke et al., 1997; and Amoah et al., 1997).

The method of introduction of nucleic acid fragments into recombination competent mammalian cells can be by any method which favors co-transformation of multiple nucleic acid molecules. Detailed procedures for producing transgenic animals are readily available to one skilled in the art, including the recitations in U.S. Pat. No. 5,489,743 and U.S. Pat. No. 5,602,307.

The practice of the present invention will employ the conventional terms and techniques of molecular biology, pharmacology, immunology, and biochemistry that are within the ordinary skill of those in the art (see Sambrook et al., Molecular Cloning—A Laboratory Manual, 2nd edition, Cold Spring Harbor Laboratory Press, 1985 or Ausubel et al., Current Protocols in Molecular Biology, John Wiley & Sons, 1994).

Other embodiments of the invention will be apparent to those skilled in the art from consideration of the specification and practice of the invention disclosed. It is intended that the specifications and examples be considered exemplary only with a true scope of the invention being indicated by the claims.

### EXAMPLE 1

### cDNA Difference Analysis of IL-9 Expressed Genes

A murine T lymphocyte cell clone TS2 was used to isolate IL-9 induced genes. TS2 is a T helper cell line derived from primary culture of murine lymphocytes as previously described (Uyttenhove et al., 1988; Louahed et al., 1995). This cell line has been shown to proliferate in response to IL-2, IL-4 or IL-9 cytokines in culture. In order to identify IL-9 specifically induced genes cDNA difference analysis was performed on mRNA from cells cultured in the presence of IL-2 or IL-9.

Cell culture and cytokines. TS2 cells were grown in DMEM medium supplemented with 10% fetal calf serum, 50 μM 2-mercaptoethanol, 0.55 mM L-arginine, 0.24 mM L-asparagine and 1.25 mM L-glutamine. This factor dependent cell line was able to grow in the presence of either IL-2, IL-4 or IL-9 without antigen or feeder cells.

cDNA synthesis. Total RNA was prepared from TS2 cells stimulated with IL-2 (200 U/ml) or IL-9 (200 U/ml) for 48 hours by the guanidine isothiocyanate method (Chomczynski et al., 1987). Polyadenylated RNA was purified from total RNA with oligo(dT) cellulose columns. Double stranded cDNA was prepared by reverse transcription using Superscript II reverse transcriptase and an oligo(dT) primer as suggested by the manufacturer (Gibco-BRL). cDNA was then prepared for cDNA difference analysis by phenol-chloroform extraction and ethanol precipitation. Products were resuspended in nuclease free water and analyzed on agarose to determine quality of products as described below.

cDNA Difference Analysis Protocol. Differential cDNA analysis of TS2 cells treated with IL-2 or IL-9 was carried out as previously described (Hubank et al., 1994), based on the genomic cDNA difference analysis procedure of Lisitsyn et al., 1993.

Oligo(dT) primers were used to generate cDNA from cytoplasmic polyadenylated mRNA isolated from TS2 cells. cDNA was digested with DpnII followed by two extractions with phenol-chloroform-isoamyl alcohol and one with chloroform-isoamyl alcohol. A glycogen carrier was added followed by precipitation with 100% ethanol. The pellet was washed with 70% ethanol, dried and resuspended in TE buffer.

For ligation of adaptors, digested cDNA was combined with desalted R-Bgl-24 (5′-AGCACTCTCCAGCCTCTCACCGCA-3′), R-Bgl-12 (5′-GATCTGCGGTGA-3′) oligos (2:1 ratio), 10×ligase buffer and water. Oligos were annealed to digested cDNA at 50° C. for one minute then cooled to 10° C. for one hour followed by addition of T4 DNA ligase and overnight incubation at 16° C. Ligation reactions were then diluted with TE buffer.

Generation of representations. Diluted ligation reaction was combined with 5×PCR buffer, dNTP nucleotide mix, water and R-Bgl-24 primer. The reaction was heated to 72° C. for three minutes to remove the 12-mer followed by addition of Taq DNA polymerase. The reactions were then incubated for five minutes at 72° C. to fill in the ends followed by a PCR cycling protocol of 20 cycles (one minute at 95° C., three minutes at 72° C.). A final extension step (ten minutes at 72° C.) was included at the end of the cycling protocol.

PCR products were extracted twice with phenol-chloroform-isoamyl alcohol, once with chloroform-isoamyl alcohol and precipitated with isopropanol. Pellets were washed with 70% ethanol and resuspsended in TE buffer. Each representation was then digested with DpnII followed by one extraction with phenol-chloroform and another with chloroform. Digested representations were precipitated with isopropanol and washed with 70% ethanol and resuspended in TE buffer. This DNA was designated the cut DRIVER.

Preparation of the TESTER. Digested representation was diluted with TE buffer and combined with 10×loading buffer loaded onto a 1.2% TAE prep gel and electrophoresed until the bromphenol blue had migrated approximately two cm. The amplicon-containing portion of the gel was excised, separating it from the digested linkers. This DNA was purified from the gel slice and resuspended in TE buffer and was designated the TESTER.

Ligation of TESTER to the J-oligos. TESTER was combined with 10×ligase buffer, water, desalted J-Bgl-24 (5′-ACCGACGTCGACTATCCATGMCA-3) and J-Bgl-12 (5′-GATCTGTTCATG-3′) oligos (2:1 ratio) then annealed to TESTER at 50° C. for one minute then cooled to 10° C. for one hour followed by addition of T4 DNA ligase and overnight incubation at 16° C. Ligation reactions were then diluted with TE buffer.

Subtractive Hybridization. The digested DRIVER representation and J-ligated TESTER representation were combined followed by extraction with phenol-chloroform. DNA was precipitated with 100% ethanol and washed twice with 70% ethanol, dried and resuspended in TE buffer. Reaction was overlaid with mineral oil and denatured for five minutes at 98° C., cooled to 67° C., followed by addition of 5 M sodium chloride and incubation for 20 hours to allow for complete hybridization.

Generation of first difference product. Mineral oil was removed and DNA was diluted with TE buffer and 5 μg/μl yeast RNA. For each subtraction setup, diluted hybridization mix was combined with 5×PCR buffer, dNTP nucleotide mix and water. The reactions were incubated at 72° C. for three minutes to remove the 12-mer, Taq DNA polymerase added, incubated another five minutes at 72° C., J-Bgl-24 primer added followed by a PCR cycling protocol of ten cycles (one minute at 95° C., three minutes at 70° C.). A final extension step (ten minutes at 72° C.) was included at the end of the cycling protocol. Reactions were extracted with phenol-chloroform-isoamyl alcohol and once with chloroform-isoamyl alcohol. A glycogen carrier was added followed by precipitation with 100% ethanol. The pellet was washed with 70% ethanol and resuspended in 0.2×TE buffer.

PCR products were then digested with mung bean nuclease for 35 minutes at 30° C. and the reaction stopped by incubation for five minutes at 98° C. in the presence of 50 mM Tris-HCl. Mung bean nuclease-treated DNA was combined with 5×PCR buffer, dNTP nucleotide mix, water and J-Bgl-24 oligo. Reactions were incubated one minute at 95° C., cooled to 80° C. and Taq DNA polymerase added followed by a PCR cycling protocol consisting of 18 cycles (one minute at 95° C., three minutes at 70° C.). A final extension step (ten minutes at 72° C.) was included at the end of the cycling protocol. PCR products were extracted twice with phenol-chloroform-isoamyl alcohol and once with chloroform-isoamyl alcohol. DNA was precipitated with isopropanol, washed with 70% ethanol and resuspended in TE buffer. This reaction product was designated the first difference product (DP1).

Change of adaptors on a difference product. DP1 was digested with DpnII, extracted twice with phenol-chloroform-isoamyl alcohol and precipitated with 100% ethanol. The pellet was washed with 70% ethanol and resuspended in TE buffer. Template DNA was combined with digested DPI, 10×ligase buffer, water, N-Bgl-24 (5′-AGGCAACTGTGCTATCCGAGGGAA-3′) and N-Bgl-12 (5′-GATCTTCCCTCG-3′) oligos (2:1 ratio) then annealed at 50° C. for one minute then cooled to 10° C. for one hour followed by addition of T4 DNA ligase and overnight incubation at 16° C.

Generation of second (DP2) and third difference product (DP3). For DP2, N-ligated DP1 was mixed with DRIVER and subtraction and amplification steps (1:800 TESTER:DRIVER ratio) were repeated as described above (J oligos were used for ligation step). For DP3, J-ligated DP2 was diluted with TE buffer containing 5 μg/μl yeast RNA. J-ligated DP2 was hybridized with DRIVER and subtraction and amplification steps (1:400,000 TESTER:DRIVER ratio) were repeated as described above to generate DP3, performing the final amplification protocol for 22 cycles.

Cloning of DP3 was achieved by digesting DP3 with DpnII, isolation on TAE prep gel as described above, purification from the excised band and cloning into pTZ19R vector. Difference products were initially characterized by: conformation of genuine difference product by probing against a blot of the original amplicons, conformation by sequencing or Northern blots to determine whether difference products originated from more than one transcript, ascertaining the frequency of cloning by probing a plasmid blot of cloned DP3 minipreps and sequencing genuine differences for potential identification via BLASTmail.

### EXAMPLE 2

### Identification of a Novel IL-9 Induced G Protein-Coupled Receptor

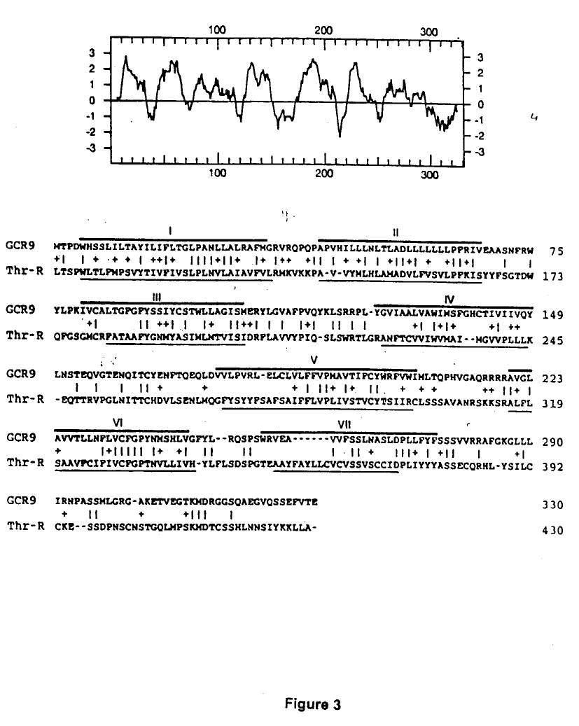

One of several cDNA identified from the cDNA difference analysis was found to be a novel cDNA that was not contained within the ENTREZ database at National Center for Biotechnology Information. A full-length cDNA was cloned from a mouse cDNA library using the fragment isolated from the difference analysis as a probe. A TS2 cDNA library was prepared by conventional methods in the pSVK3 plasmid library as previously described (Louahed et al., 1995). A 1791 base cDNA was isolated which contained an open reading frame encoding for a protein of 330 amino acids which was designated murine GCR9. FIG. 1 shows the nucleotide and amino acid sequence of the murine GCR9 cDNA including the 1.2 kilobase intron upstream of the start codon indicated by A. A nucleotide BLAST (Altschul et al., 1990) database search of the full length cDNA revealed that this cDNA is similar to several G-coupled receptor proteins. FIG. 2 shows a dendrogram of G-coupled receptor families showing protein related members to GCR9. Hydrophobicity plots of murine GCR9 demonstrate that the gene product appears to encode a polypeptide containing a seven transmembrane domain sequence motif (FIG. 3). This is a hallmark feature of other G protein-coupled receptor molecules (Larhammar et al., 1992). This data suggests that murine GCR9 is an IL-9 inducible gene involved in signal transduction.

### EXAMPLE 3

### Tissue Distribution of Murine GCR9.

Murine GCR9 gene expression was assessed in the different tissues in the FVBNJ mouse. Mice were euthanized and various organs aseptically removed and prepared for total RNA extraction using the Trizol (Gibco-BRL) method as described by the manufacturer. Total cellular RNA was fractionated by electrophoresis, transferred to Hybond-C nitrocellulose membrane (Amersham) and probed. The GCR9 probe was a 1.8 kilobase cDNA containing the complete coding sequence for GCR9 which was labeled using the RandomPrime DNA labeling kit (Boehringer Manheim). Following autoradiography, all blots were reprobed with a murine GAPDH probe to control for RNA integrity.

Analysis of a Northern blot of murine RNA derived various tissues and probed with a full length GCR9 cDNA probe revealed that GCR9 is significantly expressed in the spleen and lymph nodes and to a lesser extent in the colon and bone marrow (FIG. 4).

### EXAMPLE 4

### GCR9 is Induced In vitro by IL-9 in Murine Cells

To confirm that murine GCR9 is induced by IL-9, murine T-helper cell lines TS6 and ST2K9 were cultured in 200 U/ml murine IL-9 or human IL-2. Cells were counted and total RNA was fractionated by electrophoresis, transferred to Hybond-C nitrocellulose membrane (Amersham) and probed. The GCR9 probe was a 1.8 kilobase cDNA containing the complete coding sequence for GCR9 which was labeled using the RandomPrime DNA labeling kit (Boehringer Manheim). Following autoradiography, all blots were reprobed with a murine GAPDH probe to control for RNA integrity. The results of the in vitro experiments showed that murine GCR9 is specifically expressed in the cytokine-dependent murine cell lines TS6 and ST2K9 when cultured in the presence of IL-9 (FIG. 5). The specific induction of IL-9 is demonstrated by the fact that the gene is expressed in the presence of IL-9 but not IL-2.

To assess the inducibility of GCR9 in C57BL6 and DBA2 murine splenocytes, cultures were established and GCR9 expression was assayed by Northern blot. Splenocytes were isolated from naive DBA2 and C57BL6 mice by aseptic removal of spleens from anesthetized mice. Spleens were then aseptically minced and tissue was passed through a sterilized wire mesh sieve. Cells were resuspended in RPMI-1640 medium and washed twice with media. Cells were then resuspended in lysis buffer (4.15 g sodium chloride, 0.5 g potassium bicarbonate, 0.019 g EDTA in 500 milliliters of deionized water) to lyse red blood cells. Cells were then incubated at 37° C. for five minutes and resuspended in RPMI-1640 supplemented with 10% fetal bovine serum. Cells were centrifuged and the pellet resuspended in twenty milliliters media supplemented with 5 μg/ml of concanavalin A. Cells were harvested for RNA isolation using the guanidine isothiocyanate method described above. cDNA were generated using random hexamers (Pharmacia) and Superscript II (Gibco-BRL) as suggested by the manufacturer. Message was analyzed by PCR as previously described (Nicolaides et al., 1995). Primers used to generate murine GCR9 message were 5′-CAGACTGGCACAGTTCCTTGA-3′ (SEQ ID NO:13) and 5′-CAGATGGGTGGAGGTGTC-3′ (SEQ ID NO:14) which produce a gene product of 1,162 bases. β-actin was assayed as an internal control to measure for cDNA integrity using primers previously described (Louahed et al., 1995). Amplification conditions used were 94° C. for one minute, 60° C. for two minutes and 72° C. for two minutes with twenty and twenty-six cycles for β-actin and GCR9, respectively.

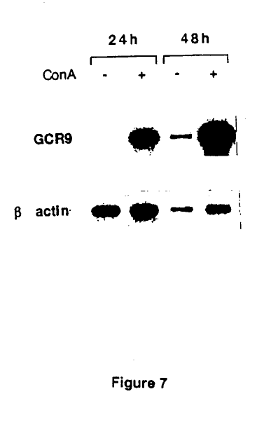

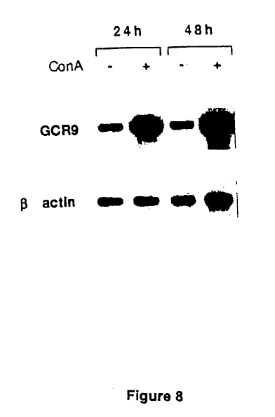

This data demonstrated that GCR9 expression is induced at twenty-four and forty-eight hours after concanavalin A stimulation in splenocytes derived from C57BL6 (FIG. 7) and DBA2 (FIG. 8). Furthermore, this data supports a role for GCR9 in the physiology of cells contained within the spleen and may have a role in signaling in splenocytes following mitogen activation.

### EXAMPLE 5

### Murine GCR9 is Induced In vitro by IL-9

The inducibility of GCR9 in primary IL-9 responsive cells was further assessed using primary mast cells derived from bone marrow. Mouse bone marrow derived mast cells (BMMC) were obtained by culturing bone marrow from BALBc mice for four weeks in RPMI-1640 medium supplemented with 0.55 mM L-arginine, 0.24 mM L-asparagine, 1.25 mM L-glutamine, 100 U/ml penicillin, 100 μg/ml streptomycin, 50 μM 2-mercaptoethanol and 20% fetal bovine serum supplemented with either one ng/ml recombinant murine IL-3 or a combination of IL-3 and 200 U/ml IL-9. FACS analysis of this population showed a homogenous staining by biotinylated IgE and no staining with anti-Mac-1, anti-Mac-2, anti-Mac-3 and anti-Thy1 antibodies.

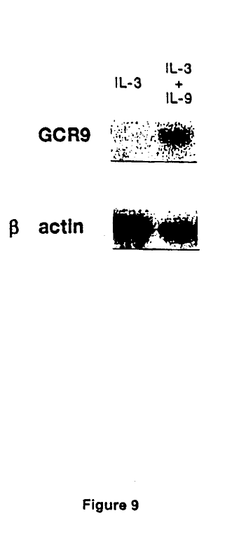

BMMC cultured in the presence of IL-3 or IL-3 plus IL-9 were harvested and RNA was extracted and reverse transcribed as described in Example 1. cDNA was analyzed by PCR for the expression of GCR9 and β-actin as described in Example 4. The data showed that GCR9 is expressed in the BMMC grown in the presence of IL-3 plus IL-9 but not in IL-3 alone (FIG. 9). This data demonstrated that GCR9 is induced in primary mast cells and may play a role in their physiology.

### EXAMPLE 6

### Murine GCR9 is Induced In vivo by IL-9

Murine GCR9 expression was assessed in vivo in both Tg5 and FVBNJ mice. FVBNJ (FVB) is the parental strain of an IL-9 transgenic strain (Tg5) which over-expresses IL-9. Generation of IL-9 trangenic mice was accomplished as previously described (Renauld et al., 1994). Mice were euthanized and the stomach was aseptically removed and prepared for total RNA extraction using the Trizol (Gibco-BRL) method as described by the manufacturer. RT-PCR was accomplished as described in Example 4. RT-PCR analysis of RNA derived from the stomach of Tg5 and FVB mice demonstrated expression of GCR9 in IL-9 transgenic mice but not in their corresponding parental strain. This data demonstrates that expression of GCR9 is induced in vivo by the presence of elevated levels of IL-9 which occur in IL-9 transgenic mice and not the parental strain.

### EXAMPLE 7

### The Cloning of the Human GCR9 Homolog

The murine GCR9 gene appears to be specifically induced in cells responsive to IL-9 by the IL-9 pathway since the same cells which are responsive to IL-2 do not express GCR9 in the presence of IL-2. In order to determine IL-9 inducibility of GCR9 in the human system, applicants used two strategies to isolate the human GCR9 homolog. First, low stringency PCR was employed to screen for homologous human GCR9 sequences. Oligodeoxynucleotides to the 5′ end of the mouse gene were employed to amplify human genomic DNA at low stringency. PCR reactions were carried out in buffers described in Example 3. The primers used were 5′-GGCACAGTTCCTTGATCCTCA-3′ (SEQ ID NO:15) and 5′-GCTCTTGGGTGMGTTCTCGT-3′ (SEQ ID NO:16) which produced a 501 base product corresponding to the N-terminus of the murine gene. Amplifications were carried out using 94° C. for 30 seconds, 60° C. for 60 seconds, 72° C. for 60 seconds for 35 cycles. Products were cloned into T-tailed vectors as described by the manufacturer (Invitrogen). Ten recombinant clones were sequenced and found to contain the same insert.

The Marathon-Ready cDNA system (Clontech, Palo Alto Calif.) was then used to clone the human GCR9 gene. This cloning system employs long-distance PCR for rapid amplification of cDNA ends (RACE) and enables the generation of a full-length cDNA. This rapid cloning system begins with double-stranded cDNA synthesis from poly A+RNA and ligation to the specially designed cDNA Adaptor. The cDNA can then be used as a template in RACE PCR to obtain the 5′ or 3′ ends or a full-length cDNA. The human GCR9 primers used in the RACE reaction were 5′-GAGCCACGTGCTGCAGTAGATGCTGCT-3′ (SEQ ID NO:17) for the 5′ reaction and 5′-CTGCCMCCTCCTGGCCCTGCGGGCCTT-3′ (SEQ ID NO:18) for the 3′ reaction. A second nested PCR was then employed to further amplify the cDNA which utilized the following nested primers: 5′-GTACCAGCGGAAGTTCGACGCAGCCTCGA-3′ (SEQ ID NO:19) for the 5′ nested reaction and 5-GTCCTTTGGTCACTGCACCATCGT-3′ (SEQ ID NO:20) for the 3′ nested reaction. The Nested PCR product was then cloned into TA cloning vector pCR21 (Clontech) and sequenced.

The RACE reaction of human cDNA with primers derived from the 5′ region of the human GCR9 gene identified a full-length cDNA (FIG. 10) whose nucleotide sequence displayed 84% homology to the murine GCR9 gene. Alignment of the deduced amino acid sequences demonstrated that human GCR9 is 84% identical to murine GCR9. The data identified the human homolog of the IL-9 inducible murine GCR9 and demonstrates an important role for this protein in cellular physiology because of its high degree of conservation between murine and human sequences.

### EXAMPLE 8

### Nucleic Acids which Hybridize to GCR9

To identify nucleic acid molecules which hybridize to the human or murine GCR9 nucleotide sequences set forth in SEQ ID NOS:1 and 3, hybridization assays are performed using any available methods to control the stringency of hybridization. Hybridization is a function of sequence identity (homology), G+C content of the sequence, buffer salt content, sequence length and duplex melt temperature (Tₘ) among other variables (See Maniatis et al., Molecular Cloning: A Laboratory Manual, Cold Spring Harbor Laboratory Press, 1982).

Hybridization analysis may be performed using genomic DNA or cDNA pools prepared from mRNA from a T lymphocyte cell line according to the procedures of Sambrook et al., Molecular Cloning: A Laboratory Manual, 2nd Edition, Cold Spring Harbor Laboratory Press, 1989, Chapter 9). Briefly, hybridization with nylon membranes is performed in 6×SSC; 0.5% SDS; 100 μg/ml denatured, sonicated salmon sperm DNA and 50% formamide at 42° C. using radiolabeled probe comprising SEQ ID NO:1 and/or SEQ ID NO:3. After hybridization, the filter is washed in 2×SSC and 0.1% SDS, followed by several washes in 0.1×SSC and 0.5% SDS at 37° C. and 0.1×SSC and 0.5% SDS at 68° C. Results are visualized by autoradiography.

Nucleic acid molecules comprising the following sequences hybridize to probe comprising SEQ ID NO:1 under the above conditions: 5′-CAGACTGGCACAGTTCCTTGA-3′ (SEQ ID NO:13) and 5′-CAGATGGGTGGAGGTGTC-3′ (SEQ ID NO:14).

Nucleic acid molecules comprising the following sequences hybridize to probe comprising SEQ ID NO:3 under the above conditions: 5′-GAGCCACGTGCTGCAGTAGATGCTGCT-3′ (SEQ ID NO:17), 5′-CTGCCMCCTCCTGGCCCTGCGGGCCTT-3′ (SEQ ID NO:18), 5′-GTACCAGCGGAAGTTCGACGCAGCCTCGA-3′ (SEQ ID NO:19) and 5-GTCCTTTGGTCACTGCACCATCGT-3′ (SEQ ID NO:20).

### EXAMPLE 9

### Tissue Distribution of Human GCR9

Human GCR9 gene expression was assessed by probing a human multiple tissue Northern blot (Clontech, Palo Alto Calif.) with a cDNA probe specific for human GCR9. The GCR9 probe was a 2.6 kilobase cDNA containing the complete coding sequence for GCR9 which was labeled using the RandomPrime DNA labeling kit (Boehringer Manheim). Following autoradiography, all blots were reprobed with a human GAPDH probe to control for RNA integrity.

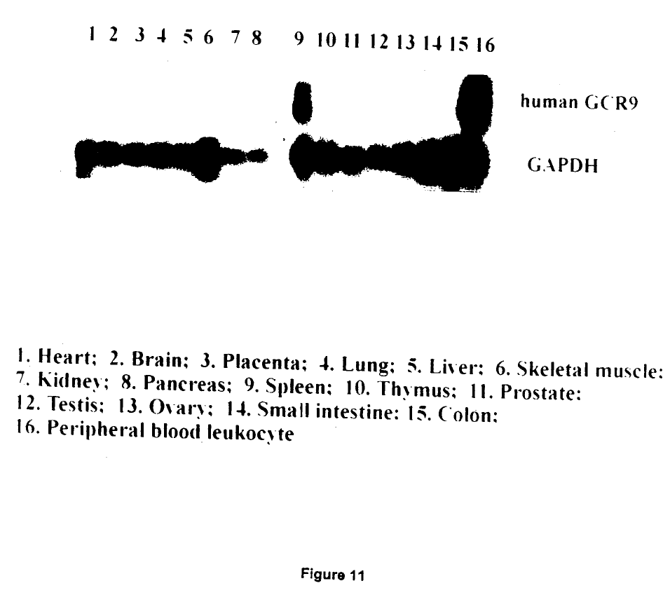

Analysis of a Northern blot of human RNA derived various tissues and probed with a full length GCR9 cDNA probe revealed that GCR9 is significantly expressed in peripheral blood leukocytes and the spleen and to a lesser extent in the colon (FIG. 11).

### EXAMPLE 10

### Human GCR9 is Induced In vitro by IL-9

To assess the ability of human GCR9 to be induced by the IL-9 pathway, primary eosinophils, and mast cells were assayed for expression levels of human GCR9. 1×10⁷ cells were isolated and washed three times with phosphate buffered saline solution and plated in twelve milliliters of RPMI-1640 medium supplemented with 0.5% bovine serum albumin and cofactors (Gibco-BRL). Following a twelve hour incubation, cells were supplemented with 50 ng/ml IL-9 for twenty-four hours. The next day, cells were harvested and total RNA extracted using the Trizol (Gibco-BRL) method described by the manufacturer. RNA was processed and reverse transcribed into cDNA as also described in Example 3. Equivalent amounts of cDNA was then amplified using the same oligdeoxynucleotide primers used for the human GCR9 isolation. β-actin was used as a control to monitor for cDNA integrity as described in Example 4. PCR amplifications were carried out at 95° C. for 30 seconds, 60° C. for 60 seconds and 72° C. for 60 seconds for 35 cycles. Reactions are electrophoresed in agarose gels and stained with ethidium bromide.

Human eosinophils and mast cells both expressed GCR9 as determined by RT-PCR. Furthermore, induction of GCR9 expression occurs in mast cells cultured in the presence of IL-9 (FIG. 12). This data demonstrated that GCR9 is induced in primary mast cells and is therefore involved in immune responses involving these cells.

### EXAMPLE 11

### Blocking of GCR9 Induction by Aminosterols in Murine Splenocytes

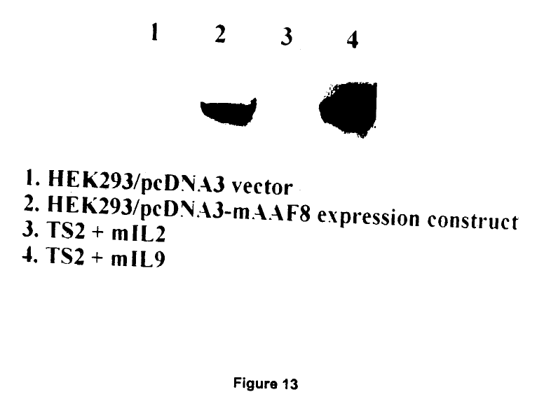

Splenocytes from the DBA2 bronchial hyperresponsive mouse were treated with aminosterol compounds to test for their ability to block the induction of GCR9 in response to mitogens. The aminosterols used were compounds identified from the liver of the dogfish shark and as a class of molecules appear to be antiproliferative. A series of aminosterols were assayed for their ability to inhibit GCR9 expression and Th2 activity in mitogen stimulated splenocytes from the DBA2 mouse. An example of the structure of these compounds is shown in FIG. 13.

Splenocytes were isolated from naive DBA2 mice by aseptic removal of spleens from anesthetized mice. Spleens were then minced and tissue was passed through a sterilized wire mesh sieve. Cells were resuspended in RPMI-1640 medium and washed twice in RPMI-1640. Cells were then resuspended in lysis buffer as in Example 3 to lyse red blood cells. Cells were incubated at 37° C. for five minutes and resuspended in RPMI-1640 supplemented with 10% fetal bovine serum. Cells were then centrifuged and the pellet resuspended in twenty milliliters of supplemented with 5 μg/ml of concanavalin A. Cell cultures were treated with 10 μg/ml of aminosterol compound for twenty-four hours. Cells were then harvested for RNA isolation using the Trizol (Gibco-BRL) method as described by the manufacturer.

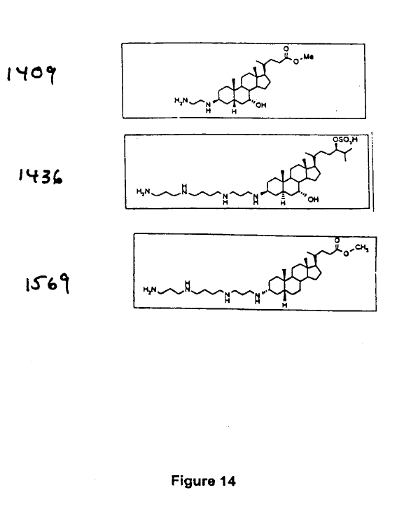

RNA derived from splenocytes treated with aminosterol compounds were reverse transcribed and message was analyzed by PCR as previously described (Nicolaides et al., 1995). Primers used to generate murine GCR9 message were sense 5′-CCAGACTGGCACAGTTCC-3′ (SEQ ID NO:17) and 5′-TGCTGTAGAAGCCGAAGCC-3′ (SEQ ID NO:18) which produce a gene product of 267 bases. β-actin was assayed as an internal control to measure for cDNA integrity using primers previously described (Nicolaides et al., 1991). Amplification conditions used were 95° C. for 30 seconds, 58° C. for 90 seconds and 72° C. for 90 seconds for 35 cycles. FIG. 14 shows the effect of the aminosterols on GCR9 expression. This data demonstrated the ability of specific aminosterols, such as 1409, to block the expression of GCR9 in vitro, while similar compounds such as 1436 and 1569 had no effect on expression.

### EXAMPLE 12

### Antibody Detection of Murine GCR9 Protein Expression

Polyclonal rabbit antisera to GCR9 was generated by immunizing rabbits with a synthetic polypeptide corresponding to residues 65-82 (RIVEAASNFRWYLPKIVC) (SEQ ID NO:6) of the murine GCR9 protein (see Harlow & Lane's Antibodies: A Laboratory Manual, Cold Spring Harbor Laboratory Press, 1988). A thousand-fold dilution of antisera was experimentally determined to be sufficient to detect GCR9 protein expression by Western blot.

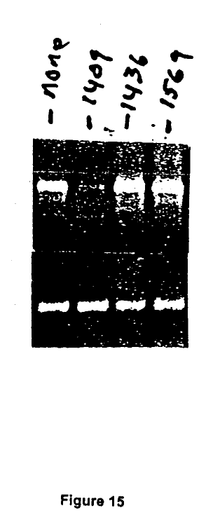

Murine GCR9 cDNA was cloned into the pCDNA3 expression vector and transfected into human embryonic kidney cells (HEK293) using the calcium phosphate method. Pooled transfected cells were selected with neomycin (3 μg/ml) and GCR9 protein expression was assayed by Western blot. HEK293 cells transfected with a expression vector encoding the murine GCR9 gene expressed significant amounts of 37 kiloDalton protein corresponding to GCR9 (FIG. 15). Results were negative for cells transfected with empty vector.

To confirm that GCR9 protein expression is induced by IL-9, the murine T-helper cell line TS2 was cultured with murine IL-9 (50 μg/ml) or IL-2 (40 μg/ml) as a negative control (R&D Systems). Cells were counted, the cell membrane fraction was isolated from equivalent number of cells and GCR9 protein induction was assayed by Western blot.

This experiment demonstrated that GCR9 is specifically expressed in the cytokine dependent murine cell line TS2 when cultured in the presence of IL-9 (FIG. 15). The specific induction by IL-9 is demonstrated by the fact that the gene is expressed in the presence of IL-9 but not IL-2 as determined by Western blot. This data demonstrates a direct effect of IL-9 on GCR9 expression, where IL-9 responsive cells produce GCR9 for intracellular signaling.

### EXAMPLE 13

### Role of GCR9 in Murine Models of Asthma: Airway Response of Unsensitized Animals

DBA2, C57BL6 or B6D2F1 mice, five to six weeks of age, are obtained from the National Cancer Institute or Jackson Laboratories (Bar Harbor ME). IL-9 transgenic mice (Tg5) and their parent strain (FVB) are obtained from the Ludwig Institute (Brussels, Belgium). Animals are housed in high-efficiency particulate filtered air laminar flow hoods in a virus and antigen free facility and allowed free access to rodent chow and water for three to seven days prior to experimental manipulation. The animal facilities are maintained at 22° C. and the light:dark cycle is automatically controlled (10:14 hour cycle).

Phenotyping and efficacy of pretreatment. To determine the bronchoconstrictor response, respiratory system pressure is measured at the trachea and recorded before and during exposure to the drug. Mice are anesthetized and instrumented as previously described. (Levitt et al., 1988; Levitt et al., 1989; Kleeberger et al., 1990; Levitt et al., 1991; Levitt et al., 1995; Ewart et al., 1995). Airway responsiveness is measured to one or more of the following: 5-hydroxytryptamine, acetylcholine, atracurium or a substance-P analog. A simple and repeatable measure of the change in peak inspiratory pressure following bronchoconstrictor challenge is used which has been termed the Airway Pressure Time Index (APTI) (Levitt et al., 1988; Levitt et al., 1989). The APTI is assessed by the change in peak respiratory pressure integrated from the time of injection until the peak pressure returns to baseline or plateau. The APTI is comparable to airway resistance, however, the APTI includes an additional component related to the recovery from bronchoconstriction.

Prior to sacrifice, whole blood is collected for serum IgE measurements by needle puncture of the inferior vena cava in anesthetized animals. Samples are centrifuged to separate cells and serum is collected and used to measure total IgE levels. Samples not measured immediately are frozen at −20° C.

All IgE serum samples are measured using an ELISA antibody-sandwich assay. Microtiter plates are coated, 50 μl per well, with rat anti-murine IgE antibody (Southern Biotechnology) at a concentration of 2.5 μg/ml in coating buffer of sodium carbonate-sodium bicarbonate with sodium azide. Plates are covered with plastic wrap and incubated at 4° C. for 16 hours. The plates are washed three times with a wash buffer of 0.05% Tween-20 in phosphate-buffered saline, incubating for five minutes for each wash. Blocking of nonspecific binding sites is accomplished by adding 200 μl per well 5% bovine serum albumin in phosphate-buffered saline, covering with plastic wrap and incubating for two hours at 37° C. After washing three times with wash buffer, duplicate 50 μl test samples are added to the wells. Test samples are assayed after being diluted 1:10, 1:50 and 1:100 with 5% bovine serum albumin in wash buffer. In addition to the test samples, a set of IgE standards (PharMingen) at concentrations from 0.8 ng/ml to 200 ng/ml in 5% bovine serum albumin in wash buffer, are assayed to generate a standard curve. A blank of no sample or standard is used to zero the plate reader (background). After adding samples and standards, the plate is covered with plastic wrap and incubated for two hours at room temperature. After washing three times with wash buffer, 50 μl of secondary antibody rat anti-murine IgE-horseradish peroxidase conjugate is added at a concentration of 250 ng/ml in 5% bovine serum albumin in wash buffer. The plate is covered with plastic wrap and incubated two hours at room temperature. After washing three times with wash buffer, 100 μl of the substrate 0.5 mg/ml o-phenylenediamine in 0.1 M citrate buffer is added to every well. After five to ten minutes the reaction is stopped with 50 μl of 12.5% sulfuric acid and absorbance is measured at 490 nm on a MR5000 plate reader (Dynatech). A standard curve is constructed from the standard IgE concentrations with antigen concentration on the x axis (log scale) and absorbance on the y axis (linear scale). The concentration of IgE in the samples is interpolated from the standard curve.

Bronchoalveolar lavage and cellular analysis are preformed as previously described (Kleeberger et al., 1990). Lung histology is carried out after the lungs are removed under anesthesia. Since prior instrumentation may introduce artifact, separate animals are used for these studies. Thus, a small group of animals is treated in parallel exactly the same as the cohort undergoing various pretreatments except these animals are not used for other tests aside from bronchial responsiveness testing. After bronchial responsiveness testing, the lungs are removed and submersed in liquid nitrogen. Cryosectioning and histologic examination is carried out in a manner obvious to those skilled in the art.

Antagonists for the murine GCR9 pathway are used therapeutically to down-regulate the functions of and assess the importance of this pathway to bronchial responsiveness, serum IgE and bronchoalveolar lavage in the sensitized and unsensitized mice. After antagonist pretreatment, baseline bronchial hyperresponsiveness, bronchoalveolar lavage and serum IgE levels relative to immunoglobulin matched controls are determined.

### EXAMPLE 14

### Role of GCR9 in Murine Models of Asthma: Airway Response of Sensitized Animals

Animals and handling are essentially as described in Example 16. Sensitization by nasal aspiration of Aspergillus fumigatus antigen is carried out to assess the effect on bronchial hyperresponsiveness, bronchoalveolar lavage and serum IgE. Mice are challenged with Aspergillus or saline intranasally (Monday, Wednesday and Friday for three weeks) and phenotyped twenty-four hours after the last dose. The effect of pretreatment by antagonists of the GCR9 pathway is used to assess the effect of down-regulating GCR9 in mice.

While the invention has been described and illustrated herein by references to various specific materials, procedures and examples, it is understood that the invention is not restricted to the particular combinations of material and procedures selected for that purpose. Numerous variations of such details can be implied as will be appreciated by those skilled in the art.

All publications, patents and patent applications herein are incorporated by reference to the same extent as if each individual publication, patent or patent application was specifically and individually indicated to be incorporated by reference.

### REFERENCES

Amoah E A and Gelaye S. Biotechnological advances in goat reproduction. J. Anim. Sci. 75, 578-585, 1997.

Alexander A G, Barnes N C and Kay A B. Trial of cyclosporin in corticosteroid-dependent chronic severe asthma. Lancet 339, 324-328, 1992.

Altschul S F, Gish W, Miller W, Myers E W and Lipman D J. Basic Local alignment search tool. J. Mol. Biol. 215, 403-410, 1990.

Amoah E A and Gelaye S. Biotechnological advances in goat reproduction. J. Anim. Sci. 75, 578-585, 1997.

Brenin D R, Talamonti M S and lannaccone P M. Transgenic technology: an overview of approaches useful in surgical research. Surg. Oncol. 6, 99-110,1997.

Burrows B, Sears M R, Flannery E M, Herbison G P and Holdaway M D. Relationship of bronchial responsiveness assessed by methacholine to serum IgE, lung function, symptoms and diagnoses in 11-year-old New Zealand children. J. Allergy Clin. Immunol. 90, 376-385, 1992.

Burrows B, Martinez F D, Halonen M, Barbee R A and Cline M G. Association of asthma with serum IgE levels and skin-test reactivity to allergens. New Eng. J. Med. 320, 271-277, 1989.

Cotton R G. Detection of single base changes in nucleic acids. Biochemical Journal 263(1), 1-10, 1989.

Chomczynski P and Sacchi N. Single-step method of RNA isolation by acid guanidinium thiocyanate-phenol-chloroform extraction. Anal. Biochem. 162,156-159, 1987.

Chu J W and Sharom F J. Glycophorin A interacts with interleukin-2 and inhibits interleukin-2-dependent T-lymphocyte proliferation. Cell. Immunol. 145, 223-239, 1992.

Clifford R D, Pugsley A, Radford M and Holgate S T. Symptoms, atopy and bronchial response to methacholine in parents with asthma and their children. Arch. Dis. Childhood 62, 66-73, 1987.

Clutter A C, Pomp D and Murray J D. Quantitative genetics of transgenic mice: components of phenotypic variation in body weights and weight gains. Genetics 143, 1753-1760, 1996.

Devos R, Plaetinck G, Cornelis S, Guisez Y, Van der Heyden J and Tavernier J. Interleukin-5 and its receptor: a drug target for eosinophilia associated with chronic allergic disease. J. Leukoc. Biol. 57, 813-819, 1995.

Doull I, Lawrence S, Watson M, Begishvili T, Beasley R, Lampe F, Holgate S T and Morton N E. Allelic association of makers on chromosome 5q and 11 q with atopy and bronchial hyperresponsiveness. Am. J. Respir. Crit. Care Med. 153, 1280-1284, 1996.

Dugas B, Renauld J C, Pene J, Bonnefoy J, Peti-Frere C, Braquet P, Bousquet J, Van Snick J, Mencia-Huerta J M. interleukin-9 potentiates the interleukin-4-induced immunoglobulin (IgG, IgM and IgE) production by normal human B lymphocytes. Eur. J. Immunol. 23, 1687-1692, 1993.

Ewart S, Levitt R C and Mitzner W. Respiratory system mechanics in mice measured by end-inflation occlusion. J. Appl. Phys. 79, 560-566, 1995.

Gergen P J and Weiss K B. The increasing problem of asthma in the United States. Am. Rev. Respir. Dis. 146, 823-824, 1992.

Gergen P J. The association of allergen skin test reactivity and respiratory disease among whites in the U.S. population. Arch. Intern. Med. 151. 487-492, 1991.

Gleich G J. The eosinophil and bronchial asthma: current understanding. J. Allergy Clin. Immunol. 85, 422-436,1990.

Godfraind C, Louahed J, Faulkner H, Vink A, Warnier G, Grencis R and Renauld J C. Intraepithelial infiltration by mast cells with both connective tissue-type and mucosal-type characteristics in gut, trachea and kidneys of IL-9 transgenic mice. J. Immunol. 160, 3989-3996, 1998.

Halonen M, Stern D, Taussig L M, Wright A, Ray C G and Martinez F D. The predictive relationship between serum IgE levels at birth and subsequent incidences of lower respiratory illnesses and eczema in infants. Am. Rev. Respir. Dis. 146, 666-670, 1992.

Houdebine L M. The production of pharmaceutical proteins from the milk of transgenic animals. Reprod. Nutr. Dev. 35, 609-617, 1995.

Hubank M and Schatz D G. Identifying differences in mRNA expression by representational difference analysis of cDNA. Nucleic Acids Research 22, 5640-5648,1994.

Johannson S G O, Bennich H H and Berg T. The clinical significance of IgE. Prog. Clin. Immunol. 1, 1-25, 1972.

Kaplan M H, Smith D I and Sundick R S. Identification of a G protein coupled receptor induced in activated T cells. J. Immunol. 151, 628-636, 1993.

Kauvar L M. Peptide mimetic drugs: A comment on progress and prospects. Nature Biotechnology 14, 709, 1996.

Kim J H, Jung-Ha H S, Lee H T and Chung K S. Development of a positive method for male stem cell-mediated gene transfer in mouse and pig. Mol. Reprod. Dev. 46, 515-526, 1997.

Kleeberger S R, Bassett D J, Jakab G J and Levitt R C. A genetic model for evaluation of susceptibility to ozone-induced inflammation. Am. J. Physiol. 258, L313-320, 1990.

Larhammar D, Blomqvist A G, Yee F, Jazin E, Yoo H and Wahlested C. Cloning and functional expression of a human neuropeptide Y/peptide YY receptor of the Y1 type. J. Biol. Chem. 67, 10935-10938,1992.

Levitt R C and Ewart S L. Genetic susceptibility to atracurium-induced bronchoconstriction. Am. J. Respir. Crit. Care. Med. 151, 1537-1542, 1995.

Levitt R C. Understanding biological variability in susceptibility to respiratory disease. Pharmacogenetics 1, 94-97, 1991.

Levitt R C and Mitzner W. Autosomal recessive inheritance of airway hyperreactivity to 5-hydroxytryptamine. J. Appl. Physiol. 67, 1125-1132, 1989.

Levitt R C, Mitzner W et al. Epression of airway hyperreactivity to acetylcholine as a simple autosomal recessive trait in mice. FASEB J. 2, 2605-2608, 1988.

Lisitsyn N, Lisitsyn N and Wigler M. Cloning the differences between two complex genomes. Science 259, 946-951, 1993.

Louahed J, Kermouni A, Van Snick J and Renauld J C. IL-9 induces expression of granzymes and high affinity IgE receptor in murine T helper clones. J. Immunol. 154, 5061-5070, 1995.

Marsh D G, Meyers D A and Bias W B. The epidemiology and genetics of atopic allergy. New Eng. J. Med. 305, 1551-1559, 1982.

Merz H, Houssiau A, Orscheschek K, Renauld J C, Fliedner A, Herin M, Noel H, Kadin M, Mueller-Hermelink H K and Van Snick J. IL-9 expression in human malignant lymphomas: Unique association with Hodgkins disease and large cell anaplastic lymphoma. Blood 78, 1311-1317, 1991.

Molinoff P et al., Goodman and Gilman's The Pharmacologic Basis of Therapeutics, MacMillan Publishing Company, New York N.Y., 1995.

Morely J. Cyclosporin A in asthma therapy: A pharmacological rationale. J. Autoimmun. 5 Suppl A, 265-269, 1992.

Mullins J J and Mullins L J. Transgenesis in nonmurine species. Hypertension 22, 630-633 1993.

Nicolaides N C, Carter K C, Shell B K, Papadopoulos N, Vogelstein B and Kinzler K W. Genomic organization of the human PMS2 gene family. Genomics 30,195-206,1995.

Nicolaides N C, Gualdi R, Casadevall C, Manzella L and Calabretta B. Positive autoregulation of c-myb expression via Myb binding sites in the 5′ flanking region of the human c-myb gene.

Mol. Cell. Biol. 11, 6166-6176,1991.

Orita M, Suzuki Y, Sekiya T and Hayashi K. Rapid and sensitive detection of point mutations and DNA polymorphisms using the polymerase chain reaction. Genomics 5, 874-879, 1989.

Petit-Frere C, Dugas B, Braquet P, Mencia-Huerta J M. Interleukin-9 potentiates the interleukin-4-induced IgE and IgG1 release from murine B lymphocytes. Immunology 79, 146-151, 1993.

Petters R M. Transgenic livestock as genetic models of human disease. Reprod. Fertil. Dev. 6, 643-645,1994.

Renauld J C, Kermouni A, Vink A, Louahed J and Van Snick J. Interleukin 9 and its receptor: Involvement in mast cell differentiation and T cell oncogenesis. J. Leukocyte Biol. 57, 353-360, 1995.

Renauld J C, van der Lugt N, Vink A, van Roon M, Godfraind C, Warnier G, Merz H, Feller A, Berns A and Van Snick J. Thymic lymphomas in interleukin 9 transgenic mice. Oncogene 9, 1327-1332, 1994.

Sarkar G, Yoon H-S and Sommer S S. Dideoxy fingerprint (ddF): A rapid and efficient screen for the presence of mutations. Genomics 13, 441-443, 1992.

Sawzdargo M, George S R, Nguyen T, Shijie X, Kolakowski L F and O'Dowd B F. A cluster of four novel human G Protein-coupled receptor genes occurring in close proximity to CD22 gene on chromosome 19q13.1. Biochem. Biophys. Res. Commun. 239, 543-547, 1997.

Schmitt E, Van Brandwijk R, Van Snick J, Siebold B and Rude E. TCGF III/P40 is produced by naive murine CD4+T cells but is not a general T cell growth factor. Eur. J. Immunol. 19, 2167-2170, 1989.

Schnieke A E, Kind A J, Ritchie W A, Mycock K, Scott A R, Ritchie M, Wilmut I, Colman A and Campbell K H. Human factor IX transgenic sheep produced by transfer of nuclei from transfected fetal fibroblasts. Science 278, 2130-2133, 1997.

Schwengel D, Nouri N, Meyers D and Levitt R C. Linkage mapping of the human thromboxane A2 receptor (TBXA2R) to chromosome 19p13.3 using transcribed 3′ untranslated DNA sequence polymorphisms. Genomics 18, 212-215, 1993.

Sears M R, Burrows B, Flannery E M, Herbison G P, Hewitt C J and Holdaway M D. Relation between airway responsiveness and serum IgE in children with asthma and in apparently normal children New Engl. J. Med. 325(15), 1067-1071, 1991.

Sheffield V C, Beck J S, Kwitek A E, Sandstrom D W and Stone E M. The sensitivity of single-strand conformation polymorphism analysis for the detection of single base substitutions. Genomics 16, 325-332, 1993.

Simoncsits A, Bristulf J, Tjornhammar M L et al. Deletion mutants of human interleukin 1 beta significantly reduced agonist properties: search for the agonist/antagonist switch in ligands to the interleukin 1 receptors. Cytokine 6, 206-214, 1994.

Uyttenhove C R, Simpson R and Van Snick J. Functional and structural characterization of P40: A mouse glycoprotein with T cell growth factor activity. Proc. Natl. Acad. Sci. USA. 85, 6934-6938, 1988.

Vink A, Renauld J C, Warnier G and Van Snick J. Interleukin 9 stimulates in vitro growth of mouse thymic lymphomas. Eur. J. Immunol. 23,1134-1138, 1993.

Zavyalov V P, Navolotskaya E V, Isaev I S et al. Nonapeptide corresponding to the sequence 27-35 of the mature human IL-2 efficiently competes with rIL-2 for binding to thymocyte receptors. Immunol. Lett. 31, 285-288, 1992.

What is claimed:

1. A purified and isolated DNA molecule having a nucleotide sequence encoding murine GCR9 or functionally equivalent fragments thereof.

2. The purified and isolated DNA molecule of claim 1 comprising the sequence of SEQ ID NO:1.

3. A purified and isolated DNA molecule having a nucleotide sequence encoding human GCR9 or functionally equivalent fragments thereof.

4. The purified and isolated DNA molecule of claim 2 comprising the sequence of SEQ ID NO:3.

5. The purified and isolated DNA molecule of claim 1 or 3, wherein said DNA molecule is genomic.

6. A chemically synthesized DNA molecule having a nucleotide sequence encoding human GCR9 or functionally equivalent fragments thereof.

7. A chemically synthesized DNA molecule having a nucleotide sequence encoding murine GCR9 or functionally equivalent fragments thereof.

8. A purified and isolated RNA molecule having a nucleotide sequence encoding human GCR9 or functionally equivalent fragments thereof.

9. A purified and isolated RNA molecule having a nucleotide sequence encoding murine GCR9 or functionally equivalent fragments thereof.

10. A purified and isolated polypeptide having an amino acid sequence comprising human GCR9 or functionally equivalent fragments thereof.

11. A purified and isolated polypeptide having an amino acid sequence comprising murine GCR9 or functionally equivalent fragments thereof.

12. A method of alleviating asthma-related disorders by administering to patients in need of such treatment an equivalent amount of a compound to down-regulate the function of human GCR9.

13. A method according to claim 12 wherein the compound comprises an aminosterol.

14. A method according to claim 13 wherein the aminosterol is 1409.

15. A method according to claim 12 wherein the compound comprises a kinase inhibitor.

16. A method according to claim 12 wherein the compound comprises the antibody of claims 30 or 31.

17. A method for detecting or diagnosing susceptibility to asthma-related disorders and certain lymphomas and leukemias associated with elevated levels of GCR9 polypeptide in a human subject comprising the steps of:
(a) measuring the level of GCR9 polypeptide in a biological sample from said human subject; and
(b) comparing the level of GCR9 polypeptide present in normal subjects, wherein an increase in the level of GCR9 polypeptide as compared to normal levels indicates a predisposition to asthma-related disorders and certain lymphomas or leukemias.

18. A method for monitoring a therapeutic treatment of asthma-related disorders or certain lymphomas or leukemias associated with elevated levels of GCR9 polypeptide in a human subject comprising; measuring the levels of GCR9 polypeptide in a series of biologic samples obtained at different time points from said subject undergoing therapeutic treatment wherein a significant decrease in said levels of GCR9 polypeptide indicates a successful therapeutic treatment.

19. A method of treating a tumor by administering to patients in need of such treatment an effective amount of a compound to down-regulate the function of human GCR9.

20. A method according to claim 19 wherein the compound comprises an aminosterol.

21. A method according to claim 20 wherein the aminosterol is 1409.

22. A method according to claim 19 wherein the compound comprises a kinase inhibitor.

23. A method according to claim 19 wherein the compound comprises the antibody of claims 30 or 31.

24. A method according to claim 19, wherein the tumor is a T cell lymphoma.

25. A method according to claim 19, wherein the tumor is a T cell leukemia.

26. A method according to claim 19, wherein the tumor is Hodgkin's lymphoma.

27. A method according to claim 19, wherein the tumor is Mycosis fungoides.

28. A method of preparing an antibody specific to an GCR9 polypeptide encoded by the DNA molecule of claims 1 or 3 or fragments thereof comprising the steps of:
(a) conjugating the GCR9 polypeptide or fragments thereof containing at least ten amino acids to a carrier protein;
(b) immunizing a host animal with said GCR9 polypeptide fragment-carrier protein conjugate admixed with an adjuvant; and
(c) obtaining antibody from the immunized host animal.

29. The method of claim 28 wherein the polypeptide is taken from SEQ ID NOS:5, 6, 7, 8, 9, 10, 11 or 12.

30. A purified and isolated antibody prepared in accordance with the method of claim 28.

31. The antibody of claim 30 wherein the antibody is monoclonal.

32. A method of quantifying a GCR9 polypeptide of claim 10 or 11 comprising the steps of:
(a) contacting a sample suspected of containing GCR9 polypeptide with an antibody that specifically binds to the GCR9 polypeptide under conditions that allow for the formation of reaction complexes comprising the antibody and GCR9 polypeptide; and
(b) detecting the formation of reaction complexes comprising the antibody and GCR9 polypeptide in the sample, wherein quantitation of the reaction complexes indicates the level of GCR9 polypeptide in the sample.

33. A method for identifying antagonists of GCR9 comprising the steps of:
(a) obtaining a cell line that is responsive to IL-9;
(b) growing said cell line in the presence of IL-9;
(c) comparing the level of GCR9 induction with that obtained with pretreatment with a possible GCR9 antagonist agent; and
(d) selecting those agents for which pretreatment diminished the characteristics.

34. The method according to claim 33 wherein the cell line is taken from the group consisting of murine TS6 cells and murine ST2K9 cells.

35. A method for identifying antagonists of GCR9 comprising the steps of:
(a) obtaining a cell line that expresses the GCR9 protein;
(b) treating said cell line with possible GCR9 antagonist agents; and
(c) selecting those agents for which treatment diminished the activity of GCR9 as measured by the level of G protein activation.

36. A method for identifying agonists of GCR9 comprising the steps of:
(a) obtaining a cell line that expresses the GCR9 protein;
(b) treating said cell line with possible GCR9 agonist agents; and
(c) selecting those agents for which treatment enhanced the activity of GCR9 as measured by the level of G protein activation.

37. Antisense DNA comprising the antisense sequence of human GCR9 or active fragments thereof.

38. A method according to claim 12 wherein the compound comprises the antisense DNA of claim 38.

39. A method according to claim 19 wherein the compound comprises the antisense DNA of claim 38.

40. An isolated nucleic acid molecule which hybridizes under stringent conditions to a nucleic acid molecule having a sequence complementary to either SEQ ID NO:1 or SEQ ID NO:3.

41. An isolated polypeptide encoded by the nucleic acid molecule of claim 40.

42. A vector comprising the DNA of claim 1.

43. A vector comprising the DNA of claim 1 operably linked to an transcriptional element, wherein said element is selected from the group consisting of prokaryotic elements or eukaryotic elements.

44. The vector of claim 43, wherein the transcriptional elements are promoters.

45. A host cell comprising the vector of claim 42, 43 or 44.

46. A method of producing GCR9 polypeptide recombinantly comprising;
A) growing the host cell of claim 45 in the appropriate nutrient medium under conditions where the polypeptide is expressed in said host cell; and
B) separating the expressed polypeptide from the host cell and nutrient medium.
